(19) United States
(12) Patent Application Publication
(10) Pub. No.: US 2021/0225499 A1
(43) Pub. Date: Jul. 22, 2021

(54) INFORMATION PROCESSING DEVICE AND PROGRAM

(71) Applicant: Masaaki TAKAYAMA, Osaka (JP)

(72) Inventor: Masaaki TAKAYAMA, Osaka (JP)

(21) Appl. No.: 17/280,270

(22) PCT Filed: Sep. 27, 2019
(86) PCT No.: PCT/JP2019/038316

§ 371 Date: Mar. 26, 2021

(30) Foreign Application Priority Data
Sep. 28, 2018 (JP) 2018-185869

(51) Int. Cl. G16H 40/20; G08B 21/04; G06Q 30/02; G06Q 10/06; G16H 10/60; G16H 20/10; G06Q 40/02

(52) U.S. Cl. CPC G16H 40/20; G08B 21/0423; G06Q 30/0281; G06Q 40/02; G16H 10/60; G16H 20/10; G06Q 10/06315

(57) ABSTRACT

A problem is to reduce the number of staff members and save labor in a reception administrative procedure of a facility that provides goods or services without requiring a customer to perform an operation on a mobile information terminal. A queue management section 101 generates queue data for patients at a medical facility and manages the queue data for each patient. An issuance controller 102 performs control to output a queue card indicating the queue data to be presented to the patients. A location management section 103 manages patient location information indicating a location in the medical facility of a patient possessing the queue card. An examination management section 104 manages an electronic chart. A procedure management section 105 manages procedural progress information indicating a progress status of a post-examination procedure including settlement of an amount of a fee determined based on information including at least the electronic chart. A monitoring section 106 performs monitoring as to whether a status of movement of the patient matches a procedure status based on the queue data, the patient location information, the electronic chart, and the procedural progress information.

### TECHNICAL FIELD

The present invention relates to an information processing device and a program capable of reducing the number of reception staff members and alleviating trouble due to a small number of reception staff members.

### BACKGROUND ART

Medical institutions, especially small ones, need staff who can perform administrative tasks such as reception and settlement. However, if the number of staff is extremely small (one person, for example), it is often difficult to handle the administrative tasks sufficiently, and errors are more likely to occur due to the increased burden on one person.

In this regard, conventional techniques have been proposed to support the administrative tasks of a medical institution. For example, Patent Literature 1 proposes a system that allows registration of a hospital at which an examination is desired, making an appointment for an examination time, settlement of a medical fee, specification of a dispensing pharmacy at which prescribed medication is desired to be received, and settlement of a fee for the prescribed medication all through a smartphone.

The system proposed in Patent Literature 1 is configured to operate in an environment where a system management host, a smartphone, a hospital host, and a pharmacy terminal are connected via the Internet. In Patent Literature 1, an appointment settlement application (hereinafter referred to as an application) is installed on a smartphone. Using the application, a hospital is registered at which an examination is desired and an appointment is scheduled at the hospital. When the actual examination is concluded after the appointment, a medical fee is displayed on the smartphone and payment is completed by clicking a payment button. When medication is prescribed, the smartphone can also be used to specify a dispensing pharmacy where the prescribed medication is to be acquired and to settle a prescription fee.

However, in the system proposed in Patent Literature 1, it is true that if a smartphone is available, registration of a hospital at which an examination is desired, making an appointment for an examination time, settlement of an examination fee, specification of a dispensing pharmacy at which prescribed medication is desired to be acquired, and settlement of a fee for the prescribed medication are possible by installing the smartphone application. However, there is a problem that this cannot be realized without a smartphone, that is, a so-called mobile information terminal.

The premise of “cannot be realized without a mobile information terminal” is that all users of a medical institution must be able to operate a mobile information terminal. However, as a practical matter, first of all, not all the users of the medical institution where the system of Patent Literature 1 is installed will have access to a mobile information terminal. Even if they have access to a mobile information terminal, they may not always carry it with them. Therefore, not all users of the medical institution can benefit from the effects of Patent Literature 1.

With the system proposed in Patent Literature 1, in addition to the above problem, the following problems may arise because, when settling an examination fee for example, the settlement is not performed face-to-face at a settlement window as before but on a smartphone (application). In other words, it is not possible to prevent patients from leaving the medical facility without going through predetermined administrative procedures after treatment, such as settling medical fees, making a subsequent appointment, and getting a prescription.

In other words, the system proposed in Patent Literature 1 is a system that allows patients with smartphones to perform administrative tasks at medical institutions on their own. Although this makes it possible to reduce the amount of labor required for administrative procedures at medical institutions, there is a risk of fraudulent or inappropriate administrative procedures occurring because most of the administrative procedures are left to the patient.

### CITATION LIST

### Patent Literature

### [Patent Literature 1]

Japanese Patent Application Laid-Open Publication No. 2016-110247

### SUMMARY OF INVENTION

### Technical Problem

In the system proposed in Patent Literature 1 as described above, it is a prerequisite that a patient carries a mobile information terminal. As such, a patient who does not carry a mobile information terminal cannot receive adequate services at a medical institution. In addition, since most of administrative procedures are left to the patients (users of the medical institution) who carry a mobile information terminal, there is a risk of fraudulent or inappropriate administrative procedures.

Taking the above situation into account, the object of the present invention is to provide a method capable of reducing the number of staff members and saving labor in the reception administrative procedure of a medical institution without requiring patients to operate a mobile information terminal.

### Solution to Problem

In order to achieve the above object, an information processing device according to an embodiment of the present invention includes:

a first management means which generates first information indicating an order in which customers receive provision of an object of goods or services at a facility where the object is provided and manages the first information for each customer;

an output means which performs control to output a medium indicating the first information to be presented to the customers;

a second management means which manages second information indicating a location in the facility of a customer among the customers possessing the medium in association with the first information;

a third management means which manages third information indicating content of the goods or services in association with the first information;

a fourth management means which manages fourth information indicating a progress status of a procedure to be performed after the provision of the object in association with the first information, the procedure including settlement of an amount of a fee for the object determined based on information including at least the third information; and

a monitoring means which performs monitoring as to whether or not a status of movement of the customer matches a procedure status based on the first information, the second information, the third information, and the fourth information.

A program according to one aspect of the present invention is a program corresponding to the information processing device according the above aspect of the present invention.

### Advantageous Effects of Invention

According to the present invention, it is possible to reduce the number of staff members and save labor in the reception administrative procedure of a medical institution without requiring a patient to operate a mobile information terminal.

### BRIEF DESCRIPTION OF DRAWINGS

FIG. 1 is a diagram illustrating an example of a configuration of a medical administrative support facility including a medical server according to one embodiment of an information processing device of the present invention.

FIG. 2 is a block diagram illustrating a hardware configuration of the medical server in the medical administrative support device in FIG. 1.

FIG. 3 is a functional block diagram illustrating an example of a functional configuration of the medical server in FIG. 2.

FIG. 4 is a flowchart depicting reception processing performed mainly by a reception terminal.

FIG. 5 is a flowchart depicting examination processing performed mainly by a medical care terminal.

FIG. 6 is a flowchart depicting settlement processing performed mainly by a procedure terminal.

FIG. 7 is a flowchart depicting appointment processing performed mainly by the procedure terminal.

FIG. 8 is a flowchart depicting prescription processing performed mainly by the procedure terminal.

FIG. 9 is a flowchart depicting monitoring processing performed mainly by an administration terminal and the medical server, and processing to activate a warning device.

FIG. 10 is a flowchart depicting reception processing performed according to a type of visitor.

FIG. 11 is a flowchart depicting the reception processing using a fingerprint authentication system.

FIG. 12 is a flowchart depicting post-examination processing performed in the medical administrative support facility.

FIG. 13 is a flowchart depicting processing when telephone reception processing is performed.

FIG. 14 is a flowchart depicting processing performed by a terminal capable of accepting the reception procedure and the post-examination procedure.

FIG. 15 is a flowchart depicting processing performed by the medical care terminal.

FIG. 16 is a flowchart depicting processing performed by the administration terminal.

FIG. 17 is a flowchart depicting processing performed by a patient terminal.

FIG. 18 is a flowchart depicting settlement processing by the medical administrative support equipment in FIG. 1 which has a charge function.

FIG. 19 is a flowchart depicting processing of the procedure terminal that constitutes the medical administrative support equipment in FIG. 1 which has a charge function.

FIG. 20 is an image diagram illustrating the content of a cloud ID managed by a cloud provided by the medical server in FIG. 1.

FIG. 21 is an image diagram illustrating a specific example of a service that can be realized by the cloud.

FIG. 22 is a flowchart depicting a settlement procedure by a patient after receiving an examination.

FIG. 23 is a flowchart depicting the settlement procedure when the patient who has received the examination is a child or when the patient has a guardian.

### DESCRIPTION OF EMBODIMENTS

An embodiment of the invention is described below using the accompanying drawings.

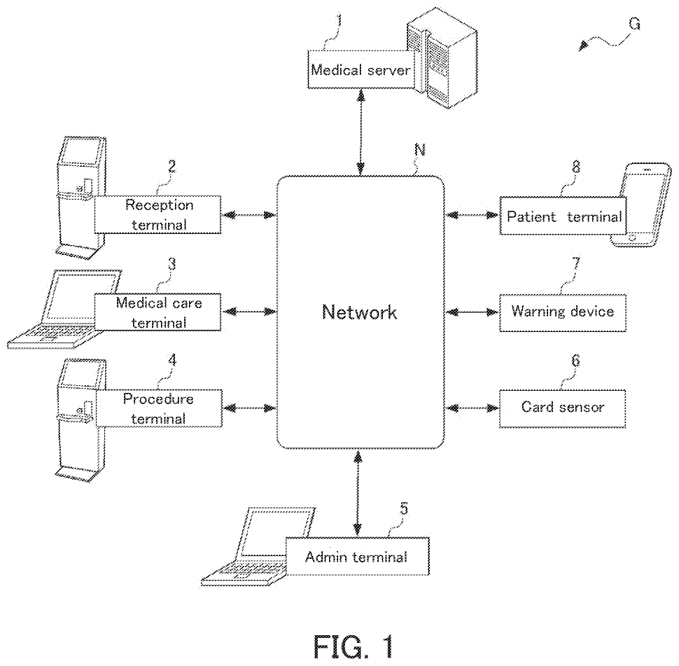

FIG. 1 is a diagram illustrating an example of a configuration of a medical administrative support facility including a medical server according to one embodiment of an information processing device of the present invention.

Medical administrative support equipment G including a medical server 1 according to the embodiment of the information processing device of the present invention solves the above-mentioned problem. That is, the medical administrative support equipment G solves the problems that may arise from utilizing a mobile information terminal of a patient to reduce the number of staff members and save labor in a reception administrative procedure of a medical institutions by using the following method.

First, the medical administrative support equipment G is installed in a target medical facility. The medical administrative support equipment G is configured to include a medical server 1, a reception terminal 2, a medical care terminal 3, a procedure terminal 4, an administration terminal 5, a card detector 6, and a warning device 7.

The reception terminal 2 identifies a visiting patient and issues a queue card indicating a turn for examination.

The medical care terminal 3 accepts input by a physician of medical care content about information related to the medical care of the visiting patient (hereinafter referred to as an “electronic chart”), which is temporarily linked to identification information that can uniquely identify the queue card issued by the reception terminal 2 (hereinafter referred to as “queue data”). The term “medical care” refers to examination and treatment by a physician.

After the medical care is finished, the procedure terminal 4 settles the fee for the current treatment, accepts an appointment for the next visit, and prints out a prescription if there is any prescribed medication.

The administration terminal 5 manages the progress of a procedure and the movement of the visiting patient, which is temporarily linked to the queue data indicated by the queue card issued by the reception terminal 2.

Card detectors 6 are installed throughout the medical facility and detect each position of one or more queue cards in the medical facility. The results of detection by the card detectors 6 are successively transmitted to the medical server 1, making it possible to grasp each position of the one or more queue cards in the medical facility in real time.

For example, the warning device 7 in the present embodiment includes a speaker installed in the medical facility, particularly near a reception counter, and a mechanism that controls unlocking and locking of the entrance and exit of the medical facility. In other words, the warning device 7 is connected to the medical server 1, and alerts a patient who has not followed the appropriate procedure with the speaker or the like and locks the entrance and exit to prevent the patient from leaving the medical facility. When the warning device 7 is activated, the administration terminal 5 can also monitor the situation.

The warning device 7 is not limited to the above. For example, the queue card itself can be made a part of the warning device 7 by configuring the queue card to do things such as emitting light, vibrating, displaying text, and emitting sound. In addition, in conjunction with these points, it is possible to separately notify a patient using items such as a telephone number and an email address of the patient registered at the time of issuance of a patient registration card even in the unlikely event that the patient leaves the medical facility without following predetermined steps or a predetermined procedure.

It is preferable that the warning device 7 be capable of informing a patient who has not followed the predetermined steps or procedure and making the patient complete the predetermined steps or procedure in a timely manner. It is even more preferable that the warning device 7 be capable of preventing a patient who has not followed the predetermined steps or procedure from leaving the medical facility and making the patient complete the predetermined steps or procedure. The configuration of the warning device 7 is not particularly limited as long as these points can be realized.

In the medical administrative support equipment G, the medical server 1, the reception terminal 2, the medical care terminal 3, the procedure terminal 4, and the administration terminal 5 are communicably connected to each other via a network N.

The medical administrative support equipment G can be further connected to the warning device 7 that can be controlled by the medical server 1. In this case, the medical server 1 can monitor the movement of each queue card and the processing status of the procedure terminal 4, detect that the predetermined steps or the predetermined procedure have not been followed, and activate the warning device 7.

In addition, the medical administrative support equipment G is communicably connected to items such as external receipt computers, various credit card companies, and bank host computers via the network N.

Here, “receipt” refers to the medical fee statement prepared for each patient every month based on the content of the patient's chart, and “receipt computer” refers to an information processing device that prepares the receipt. The medical facility can obtain medical fees by submitting receipts to a given payment organization (the National Health Insurance Federation, for example).

The reception terminal 2 reads the patient's data stored in the medical server 1 based on the patient's possession or the patient themselves, temporarily associates it with the queue data, and issues a queue card. The medical care terminal 3 receives the patient's electronic chart transmitted in the order in which the queue card was issued, and processes the input of examination items by a physician. The procedure terminal 4 calculates a fee based on the electronic chart transmitted after the examination by the physician, and also processes a settlement and flags the settlement as complete. When the patient needs a subsequent visit, the procedure terminal 4 displays information on date and time availability on a screen thereof and allows the patient to enter the date and time they wish to make an appointment. After this is completed, the procedure terminal 4 flags the appointment as complete. The medical server 1 monitors queue card status and manages flag status in the procedure terminal 4. In addition, the medical server 1 controls the warning device 7 to activate a warning when the queue card status does not match the processing status in the procedure terminal 4.

In addition to the above, the medical administrative support equipment G may employ, for example, a queue card including a so-called radio frequency identifier (RFID) function (chip) capable of contactless communication by itself. In this case, when a card detector 6 is provided around the reception terminal 2, the medical care terminal 3, and the procedure terminal 4 in the medical facility, for example, the card detector 6 can detect that the patient has passed the reception terminal 2, the medical care terminal 3, and the procedure terminal 4 in the stated order, and the medical server 1 can acquire these results. This allows the medical server 1 to monitor the movement of the queue card and that the procedure is flagged as complete at the administration terminal 5 based on the results of detection by the card detector 6.

The operation of the medical administrative support equipment G with the above configuration and the processing steps of a medical administrative support program of the present invention are described. In the medical administrative support program of the present invention, the main program is installed on the medical server 1, and client programs are installed on the reception terminal 2, the medical care terminal 3, the procedure terminal 4, and the administration terminal 5 which are connected to the medical server 1 via the communication line network N.

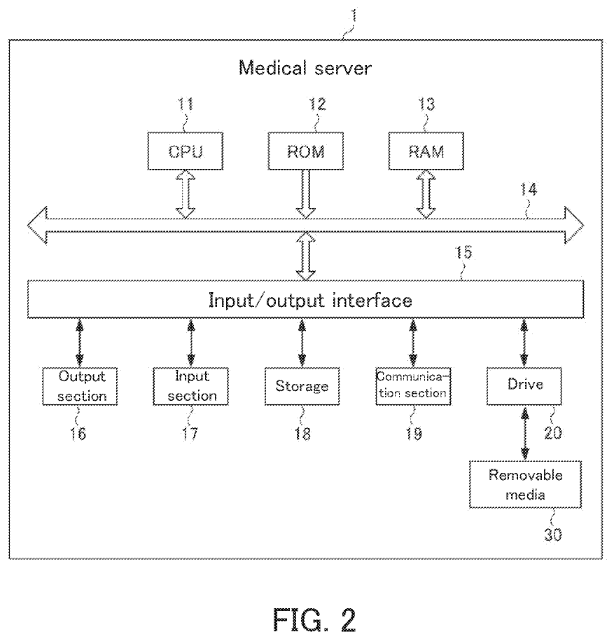

FIG. 2 is a block diagram of the hardware configuration of the medical server in the medical administrative support device in FIG. 1.

The medical server 1 includes a central processing unit (CPU) 11, read-only memory (ROM) 12, random-access memory (RAM) 13, a bus 14, an input/output interface 15, an output section 16, an input section 17, storage 18, a communication section 19, and a drive 20.

The CPU 11 performs various processing according to various programs recorded in the ROM 12 or loaded into the RAM 13 from the storage 18.

The RAM 13 also appropriately stores items such as data necessary for the CPU 11 to perform the various processing.

The CPU 11, the ROM 12, and the RAM 13 are connected to each other via the bus 14. The input/output interface 15 is also connected to the bus 14. The input/output interface 15 is connected to the output section 16, the input section 17, the storage 18, the communication section 19, and the drive 20.

The output section 16 includes items such as various liquid-crystal displays and outputs various information.

The input section 17 includes items such as various hardware and inputs various information.

The storage 18 includes items such as a hard disk and dynamic random-access memory (DRAM) and stores various data.

The communication section 19 controls communication between the medical server 1 and other devices (for example, the reception terminal 2, the medical care terminal 3, the procedure terminal 4, the administration terminal 5, the card detector 6, the warning device 7, and a patient terminal 8 in FIG. 1) via the network N which includes the Internet.

The drive 20 is provided as needed. Removable media 21, such as a magnetic disk, an optical disk, a magneto-optical disk, or semiconductor memory, can be appropriately attached to the drive 20. The program read out from the removable media 21 by the drive 20 is installed in the storage 18 as necessary. The removable media 21 can also store various data stored in the storage 18 in the same manner as the storage 18.

The hardware configuration of the reception terminal 2, the medical care terminal 3, the procedure terminal 4, the administration terminal 5, and the patient terminal 8 may be basically the same as that of the medical server 1, but may also additionally have the following hardware configuration.

The reception terminal 2 has a touch panel and can be configured specifically for reading card information and input/output for issuing a queue card.

The medical administrative support program according to an embodiment of the program of the present invention is installed in the hard disk of each of the medical care terminal 3 and the administration terminal 5.

The procedure terminal 4 has a touch panel and can be configured to handle money and cards like a so-called ATM device. The procedure terminal 4 can also be configured to allow a patient to enter the desired date and time of their next appointment, and to print out items such as prescriptions and receipts.

The collaboration of the various hardware and software of the medical server 1, the reception terminal 2, the medical care terminal 3, the procedure terminal 4, the administration terminal 5, and the warning device 7 in FIG. 2 enables the performance of a series of processing as described below.

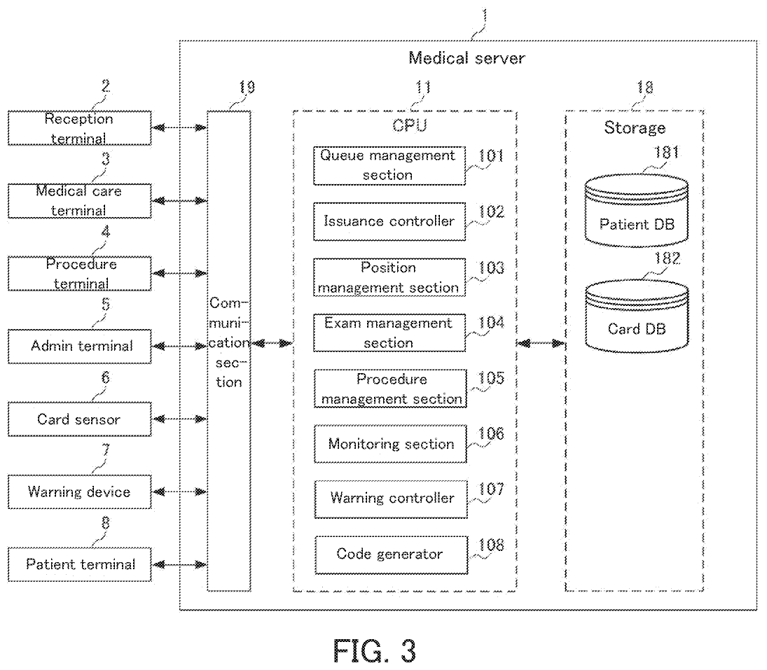

FIG. 3 is a functional block diagram illustrating an example of the functional configuration of the medical server in FIG. 2.

The CPU 11 of the medical server 1 functions as a queue management section 101, an issuance controller 102, a location management section 103, an examination management section 104, a procedure management section 105, a monitoring section 106, a warning controller 107, and a code generator 108.

A patient DB 181 and a card DB 182 are provided in one area of the storage 18 of the medical server 1.

In the patient DB 181, information about each of one or more patients (hereinafter referred to as “patient data”) is recorded and managed.

In the card DB 182, the queue data that enables specification of each of one or more queue cards is recorded and managed.

The queue management section 101 generates queue data indicating the order of patients to be examined by a physician at the medical facility, and manages the queue data corresponding to each patient.

Specifically, the queue management section 101 manages the correspondence between the patient data recorded and managed in the patient DB 181 and the queue data recorded and managed in the card DB 182.

The issuance controller 102 performs control of issuance of a queue card to be carried in the medical facility by a patient who is to be examined by a physician in the medical facility.

Specifically, the issuance controller 102 performs control of issuance of a queue card from the reception terminal 2 based on the operation on the reception terminal 2 by a patient who is visiting the medical facility. When the patient receives the queue card issued from the reception terminal 2, the patient moves within the medical facility while carrying the queue card.

The location management section 103 manages information indicating the location of the patient carrying the queue card in the medical facility (hereinafter referred to as “patient location information”).

Specifically, the location management section 103 generates and manages patient location information based on the results of detection of the queue card by the card detectors 6 installed throughout the medical facility.

The examination management section 104 manages the electronic chart by associating it with the queue data.

Specifically, the examination management section 104 acquires the electronic chart generated based on the content of the input operation by the physician to the medical care terminal 3, and manages the electronic chart by associating it with the queue data. A generated electronic chart is recorded and managed for each patient in the patient DB 181.

The procedure management section 105 manages the amount of the patient's medical fee and the progress of the payment of the patient's medical fee.

Specifically, the procedure management section 105 manages, in association with the queue data, information indicating the amount of the patient's medical fee (hereinafter referred to as “settlement data”) and information indicating the progress of various procedures (hereinafter referred to as “procedural progress information”) including the payment of the medical fee by the patient that is performed after the examination (hereinafter referred to as a “post-examination procedure”).

Specifically, the procedure management section 105 manages information such as that indicating whether or not a prescription has been issued and whether or not an appointment for a subsequent examination has been made in addition to information indicating whether or not the medical fee has been paid as the procedural progress information in association with the queue data.

In addition, the procedure management section 105 manages the correspondence between the queue data and the settlement data indicating the amount of the medical fee determined based on a receipt.

In addition, the procedure management section 105 manages the correspondence between information indicating the amount of deposit money deposited (charged) by the patient and identification information such as an ID that can uniquely specify the patient (hereinafter referred to as a “patient ID”).

Based on the queue data, the patient location information, the electronic chart, and the procedural progress information, the monitoring section 106 monitors whether or not the movement status of the patient matches the procedure status.

Specifically, the monitoring section 106 monitors the movement status of the patient and the flag status indicating the procedure status based on various data managed for each patient by the queue management section 101, the location management section 103, the examination management section 104, and the procedure management section 105.

The warning controller 107 performs control to give a predetermined warning to a corresponding patient when the procedural status of the patient does not match the movement status of the patient as a result of monitoring by the monitoring section 106.

That is, when the results of the monitoring by the monitoring section 106 reveal that there is an inconsistency between the procedural progress status and the actual position of the patient in the medical facility, there is a possibility that the patient has made a mistake or is committing fraud. For this reason, the warning controller 107 implements a warning through the warning device 7 as described above based on the results of the monitoring by the monitoring section 106.

The code generator 108 generates an identifier (a QR code (registered trademark), for example) capable of uniquely specifying the settlement data managed by the procedure management section 105.

As described above, the program executed in the medical server 1 is substantially programmed to control items such as the various terminals that constitute the medical administrative support equipment G, to send and receive various data, and to control the implementation of a warning by the warning device 7 based on the detection of the position of a queue card by the card detector 6 and the completion flag status of the various procedures.

The following is a detailed description of the various processes performed by the main program included in the medical administrative support program installed on the medical server 1 and the client program installed on each of the terminals that constitute the medical administrative support equipment G.

In the description of the present embodiment, it is assumed that there is only one person in charge of reception operation at the medical facility: an administrative staff member who operates the administration terminal 5.

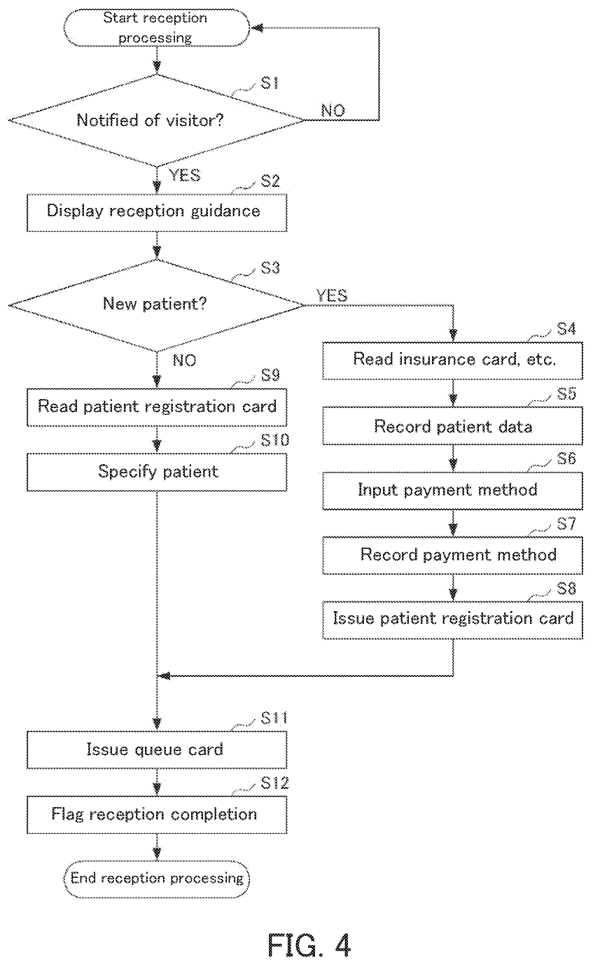

FIG. 4 is a flowchart depicting reception processing performed mainly by the reception terminal.

When a patient arrives, the reception terminal 2 installed at the entrance of the medical facility starts the reception processing. “Reception processing” refers to processing performed to receive a patient who has arrived at the medical facility for an examination.

Specifically, the reception terminal 2 detects the arrival of a patient using a motion sensor or the like (Step S1). When the reception terminal 2 detects the arrival of a patient (that is, YES in Step S1), the display of the reception terminal 2 displays “guidance display for reception procedure”, for example (Step S2).

In the “guidance display for reception procedure” of the reception terminal 2, a screen to select new patient or returning patient is displayed. When it is the patient's first visit (that is, YES in Step S3), items such as the patient's insurance card is read by a reading means (scanning function, for example) of the reception terminal 2 (Step S4). Then, the reception terminal 2 sends the read data of the insurance card or the like to the medical server 1, and the medical server 1 records patient data including items such as the information written on the insurance card in the patient DB 181 (Step S5).

The reception terminal 2 then switches from the screen that prompts the reading of the insurance card or the like to a selection screen for a means of payment, where the patient is asked to enter a means of payment (Step S6). For example, any one of the following can be selected as a means of payment: cash payment, agency payment by a credit card company, or payment by debiting from a bank account.

Specifically, for example, when cash payment is selected, the reception terminal 2 sends information to that effect to the medical server 1. The medical server 1 then records data indicating the means of payment in association with the patient data recorded in the patient DB 181 in Step S5 (Step S7).

When agency payment by a credit card company is selected, the patient causes the reception terminal 2 to read a credit card and enters various information such as the cardholder. The reception terminal 2 sends the data indicating the means of payment including the information entered by the patient to the medical server 1. The medical server 1 records the data indicating the means of payment in association with the patient data recorded in the patient DB 181 in Step S5 (Step S7).

When payment by debiting from a bank account is selected, the patient causes the reception terminal 2 to read a bank cash card and enters various information such as the name of the account. The reception terminal 2 sends the data indicating the means of payment including the information entered by the patient to the medical server 1. The medical server 1 records the data indicating the means of payment in association with the patient data recorded in the patient DB 181 in Step S5 (Step S7).

After Steps S4 and S7, the reception terminal 2 issues a patient registration card (Step S8). This patient registration card is card-shaped and is issued from an issuing slot of the reception terminal 2, and is labeled with the patient's name and a predetermined ID. If the patient does not take the output patient registration card within a predetermined period of time, the reception terminal 2 emits a warning sound and displays that fact on the screen until the patient takes the patient registration card. When the patient takes the patient registration card, the warning sound stops and the screen returns to the initial screen.

By contrast, if it is not the first visit in Step S3 (that is, NO in Step S3) but a repeat visit, the patient inserts a previously issued patient registration card into the reception terminal 2. Then, at least part of the information displayed on the surface of the patient registration card is read by the reception terminal 2 or entered by the patient (Step S9). Then, the reception terminal 2 sends the read or input data to the medical server 1 and makes an inquiry to specify the patient. When the patient is specified as a result of this inquiry (Step S10), a queue card is issued (Step S11).

In the present embodiment, the reception terminal 2 is configured to supply the patient with a card-shaped patient registration card, but this is only an example. For example, an object capable of contactless data transmission and reception and capable of being affixed such as an IC chip can be supplied as a patient registration card (or similar object). In this case, the object can be attached to an item carried by the patient, such as a mobile phone, a wallet, or a bag. Such a configuration can prevent the patient from forgetting to bring a card-type patient registration card and not being able to receive an examination smoothly, or from being treated as a new patient and burdened with the corresponding procedure and payment.

When the reception terminal 2 issues a queue card indicating the order of examination to the visiting patient, the medical server 1 sets a flag indicating that the reception has been completed (Step S12), and data to that effect is sent to the medical care terminal 3, the procedure terminal 4, and the administration terminal 5. Through the above, the reception processing ends.

Once the queue card is issued at the reception terminal 2, the medical server 1 monitors the movement of the position of the queue card detected by the card detector 6 and flags indicating the completion of the procedure at the reception terminal 2, the medical care terminal 3, and the procedure terminal 4. Through the above, when the procedure is not performed in a predetermined order, the medical server 1 performs control of processing by the warning device 7 and sends data indicating the current status to the administration terminal 5. As a result, the administration terminal 5 can monitor the movement of the position of the queue card and the flag status of procedure completion.

In other words, by looking at the screen of the administration terminal 5, the administrative staff member (one person in the present embodiment) in charge of the reception operation can easily see the movement of the patient's position (that is, the position of the queue card) and which procedure the patient has completed at the moment. This can also prevent the patient from walking out of the medical facility out of order, and for example without completing the procedure for payment of medical fees.

A patient to which the queue card has been issued by the reception terminal 2 waits in the medical facility until it is their turn to be examined. When their turn comes, they enter the examination room to be examined by a physician.

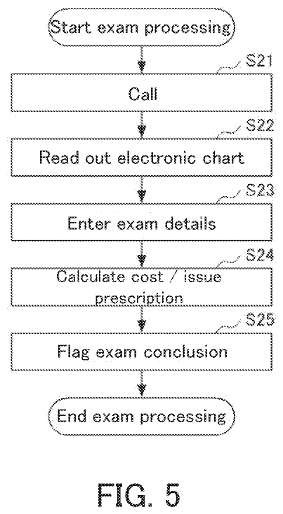

Next, the flow of processing mainly performed by the medical care terminal 3 that is operated by the physician performing the examination is described with reference to FIG. 5.

FIG. 5 is a flowchart depicting examination processing performed mainly by the medical care terminal.

The medical care terminal 3 operated by the physician who examines the patient displays the number on the queue card of the patient who is to be examined next. When the physician who has finished examining the previous patient performs a predetermined operation on the medical care terminal 3, the next patient who has the queue card with the number displayed on the medical care terminal 3 is called (Step S21).

The patient is notified of this call by the display of the number on the queue card on a predetermined display device installed in front of the examination room, or by the flashing of the displayed number. The display device is configured to display the numbers on the queue cards of patients waiting for their turn to be examined in a predetermined order. The numbers on the queue cards of one or more patients waiting for their turn to be examined are also displayed in a list on each of the display screens of the medical care terminal 3 and the administration terminal 5.

When the physician who has summoned a patient into the examination room starts the examination, an electronic chart managed in the medical server 1 in association with the queue data is read out and displayed on the medical care terminal 3 in a state that allows input (Step S22). After the examination is performed by the physician, the physician who performed the examination inputs the content of the examination into the electronic chart by operating the medical care terminal 3 (Step S23).

The medical server 1 causes an external receipt computer, for example, to calculate the medical fee based on the content of the electronic chart in which the content of the examination has been entered by the physician. The physician also prescribes medication as needed by operating the medical care terminal 3 (Step S24). When the physician completes the processing up to Step S24 and indicates that the medical examination has been concluded at the medical care terminal 3, the medical server 1 sets a flag indicating that the examination of the patient possessing the queue card has been completed (Step S25). Then, the data indicating this is sent to the procedure terminal 4 and the administration terminal 5.

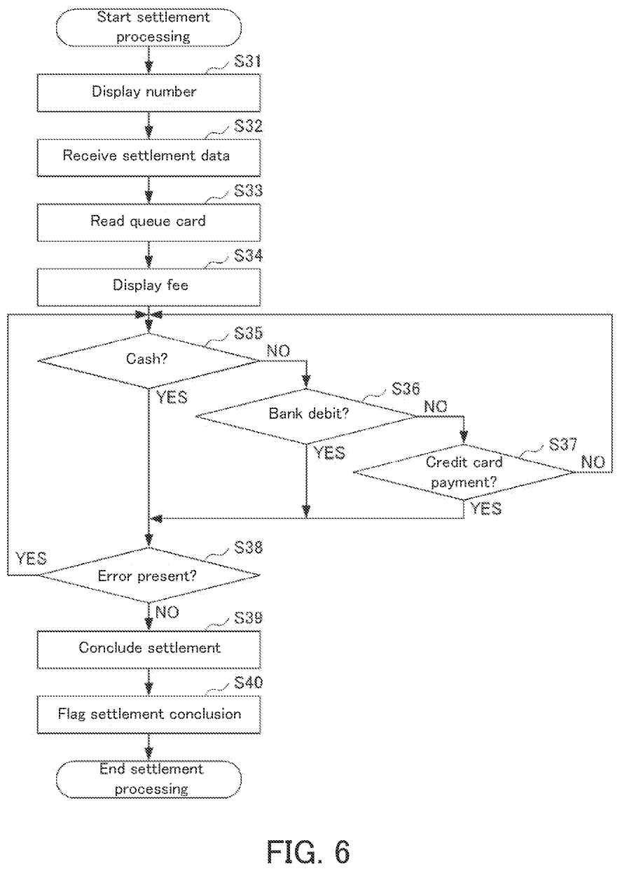

Next, the flow of processing mainly performed by the procedure terminal 4 operated by the patient after the examination is described with reference to FIG. 6.

FIG. 6 is a flowchart depicting settlement processing executed mainly by the procedure terminal.

When processing to calculate the medical fee is completed after the physician has completed the medical treatment for the patient, the medical server 1 performs control to display the number on the patient's queue card on the display screen of the procedure terminal 4 (Step S31). In addition, the medical server 1 transfers the settlement data of the target patient to the procedure terminal 4. The procedure terminal 4 receives the settlement data transferred from the medical server 1 (Step S32).

When the target patient loads the queue card as an operation to the procedure terminal 4 (Step S33), the fee based on the content of the current medical treatment is displayed (Step S34). In addition, guidance about the means of payment is also displayed.

The display of guidance about the means of payment is displayed on the screen, with characters indicating one means of payment, among cash payment, payment by debiting from a bank account, and agency payment by a credit card company, selected by the patient at the time of issuance of the patient registration card displayed first and slightly larger. For example, in the case of cash payment (that is, YES in Step S35), by operating a cash display on the procedure terminal 4, a cash slot of the procedure terminal 4 is opened and cash is inserted therein. If there is no error such as an insufficient amount or unreadable bills or coins (NO in Step S38), the settlement is completed (Step S39).

By contrast, if the payment is debited from a bank account for example (that is, YES in Step S36), the medical server 1 communicates with the host computer of an external bank for confirmation. If the payment can be debited, that is, if there is no error (NO in Step S38), the settlement is completed (Step S39).

Furthermore, if the payment is performed by credit card (that is, YES in Step S37), the medical server 1 communicates with the host computer of an external credit card company for confirmation. If the payment agent is available, that is, if there is no error (that is, NO in Step S38), the settlement is completed (Step S39).

As described above, when the settlement is completed by any method at the procedure terminal 4 (Step S39), the medical server 1 sets a flag indicating that the settlement procedure is completed for the patient with the queue card (Step S40), and this data is sent to the administration terminal 5.

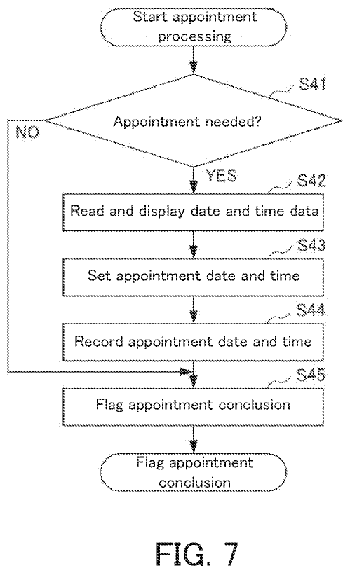

FIG. 7 is a flowchart depicting appointment processing performed mainly by the procedure terminal.

After the settlement processing depicted in FIG. 6 as above, the procedure terminal 4 switches from the display for settlement to a display for a subsequent appointment, and the screen displays whether or not a subsequent appointment is necessary (Step S41). When an operation is performed indicating that a subsequent appointment is unnecessary (that is, NO in Step S41), the processing advances to Step S45. When an operation is performed indicating that a subsequent appointment is necessary by contrast (that is, YES in Step S41), the procedure terminal 4 reads date and time data from the medical server 1 and displays a calendar on the screen for specification of the date and time (Step S42).

In displaying the calendar, when any time in a day is filled, the procedure terminal 4 will change the color of the day to indicate as such. When there is available time, the procedure terminal 4 changes the color to a different color than that of the aforementioned filled time to indicate as such. When the patient operates a day with available time, the procedure terminal 4 displays the time for that day. In the calendar display, the color of the time slots that are filled and the time slots that are not filled may be changed, or only the time slots of the physician in charge may be listed and displayed. The patient operates the procedure terminal 4 to set the date and time of the next appointment (Step S43).

When the patient sets the date and time for the next appointment by operating and confirming the available time on the procedure terminal 4, the procedure terminal 4 sends the updated appointment date and time data to the medical server 1. The medical server 1 then associates the patient data associated with the queue card with the appointment date and time data, and updates and records both the patient data and the appointment date and time data (Step S44).

When the appointment procedure is completed at the procedure terminal 4, the medical server 1 sets a flag indicating that the appointment procedure has been completed for the patient with the queue card (Step S45), and this data is sent to the administration terminal 5.

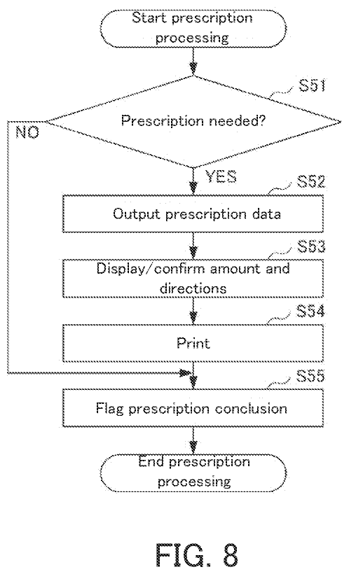

FIG. 8 is a flowchart depicting prescription processing performed mainly by the procedure terminal.

If prescription data based on electronic data created by the medical care terminal 3 via the medical server 1 is present (that is, YES in Step S51), the procedure terminal 4 switches from the appointment screen to a screen indicating that a prescription is present, reads the prescription data from the medical server 1 (Step S52), and displays it.

When the prescription data is displayed on the screen, the procedure terminal 4 displays precautions related to the medication and displays confirmation data of the dosage and directions to the patient (Step S53). The procedure terminal 4 prints the data after the patient performs a confirmation operation in Step S53 (Step S54).

When the procedure related to the prescription is completed at the procedure terminal 4, the medical server 1 sets a flag indicating that the procedure related to the prescription has been completed for the patient with the queue card and that all procedures at the procedure terminal 4 have been concluded (Step S55), and this data is sent to the administration terminal 5.

When prescription data is not present (not necessary) (that is, NO in Step S51), the processing advances to Step S55.

If the queue card is for example placed in a collection box of the reception terminal 2 after Step S55, the association between the data of the number on the queue card and the patient card is canceled, and the patient is allowed to leave the medical facility.

Next, the processing of the administration terminal 5, the medical server 1, and the warning device 7 as above is described.

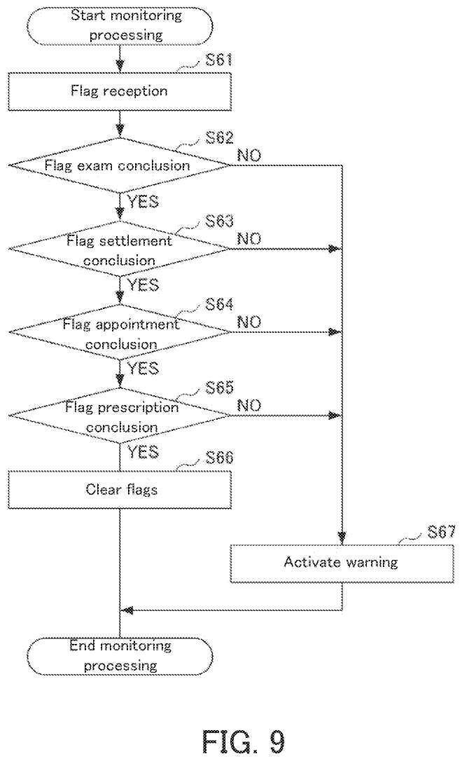

FIG. 9 is a flowchart depicting monitoring processing mainly performed by the administration terminal and the medical server, and processing to activate the warning device.

As described above, the queue card has a so-called RFID function that enables contactless communication. If a patient moves while carrying the queue card, the movement will be detected by transmitting and receiving devices, and the current position of the patient can be grasped by the administration terminal 5 via the medical server 1. By contrast, the procedural status can be grasped by the completion flag of each procedure of reception, medical treatment, settlement, appointment, and prescription (examination completion) of the patient associated with the queue card via the medical server 1.

The administration terminal 5 allows the reception staff member to monitor, via the medical server 1, the movement of the queue card and the procedure completion flags for the patient associated with the queue card. The administration terminal 5 is configured to display, when the medical server 1 detects a patient who has not followed the predetermined procedure, that fact on the screen and to cancel stoppage of the activated warning device 7.

In other words, if the number of reception staff members is reduced to one in the case of the present example, the most difficult matter is avoiding the trouble of not being able to grasp the patient's movement and the actual procedural status, such as the patient leaving the medical facility after the examination without settling.

The medical server 1 detects a queue card using the transmitting and receiving devices installed around the reception terminal 2, the medical care terminal 3, and the procedure terminal 4, and buffers the detection results. Then, after a queue card has been issued and the flag of reception completion has been set (Step S61), the medical server 1 compares the point at which the corresponding queue card was detected with the flagged procedure from Step S62 onward. When the queue card is detected at a point past an unflagged procedure (that is, NO in Steps S62 to S65), the medical server 1 activates the warning device 7.

When the medical server 1 activates the warning device 7, an announcement such as “the person with number 00 has not completed the procedure for 00” is played from a speaker in the medical facility, for example, along with the number of the queue card, and this information is displayed on the administration terminal 5. By doing this first of all, it is possible even for only one reception staff member to grasp that the movement of the queue card has not followed the predetermined order.

In particular in the present example, when the corresponding queue card is detected at the queue card detection point around the administration terminal 5 before the settlement completion flag (Step S63), the appointment completion flag (Step S64), or the prescription completion (procedure completion) flag (Step S65) is set, the door of the entrance and exit of the medical facility will not open and the patient is not allowed to leave the medical facility.

In addition, since the administration terminal 5 can grasp a situation in which the movement of the queue card is not following the predetermined order, the medical server 1 can cause the administration terminal 5 to display an instruction screen so that the person operating the administration terminal 5 can run to the patient for guidance or caution in some cases.

As such, the present invention can reduce the number of reception staff members by processing the procedures such as appointment and settlement by the procedure terminal 4, and can alleviate problems caused due to a small number of reception staff members because the administration terminal 5 manages the movement and procedural transition of a patient associated with a queue card and the warning device 7 informs the patient and the administration terminal 5 when the procedure is not followed.

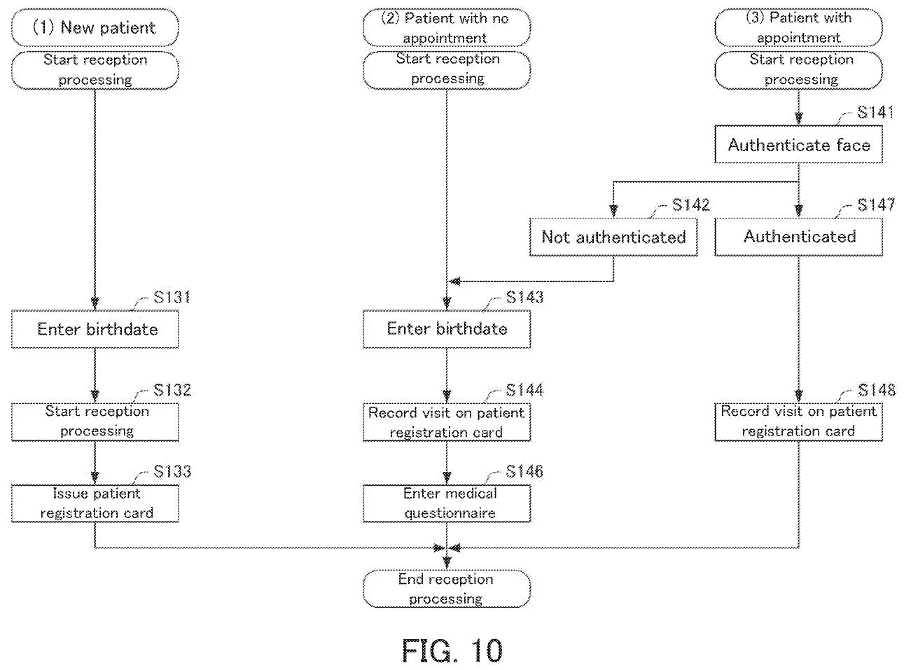

FIG. 10 is a flowchart depicting the reception processing performed according to the type of visitor.

An access management system can also use a facial authentication system to limit access at the entrance of a medical institution or an examination room. As depicted in FIG. 10, three types of visitors are assumed: (1) a new patient (first-time patient); (2) a patient who already has a patient registration card but has not visited for a while and does not have an appointment (patient without appointment); and (3) a patient who has an appointment for an examination (patient with appointment).

In the case of the type of visitor described above in (1), when the reception processing is started, the input of the visitor's date of birth is accepted (Step S131), and a basic medical questionnaire and information about the visitor are registered in a terminal. At that time, in order to enable the visitor to use the facial authentication system on a subsequent visit, an image of the visitor's face is captured (Step S132), and a patient registration card is issued based on the registered patient data (Step S133). This completes the reception processing.

In the case of the type of visitor described above in (2), the input of the visitor's date of birth is accepted (Step S143), the entrance door of the medical institution is unlocked, whether or not the visitor is a visitor present as a patient in patient data is determined using the patient registration card, and the visitor is registered (Step S144). After that, the visitor enters the content of the medical questionnaire (Step S146). Through the above, the reception processing ends.

In the case of the type of visitor described above in (3), the entrance door of the medical institution is unlocked by the facial authentication system (Steps S141 and S147), and the visitor is registered using the patient registration card (Step S148). On the other hand, when smooth performance of the facial authentication system fails (Step S142), the input of the visitor's date of birth is accepted (Step S143), the entrance door of the medical institution is unlocked, whether or not the visitor is a visitor present as a patient in patient data is determined using the patient registration card, and the visitor is registered (Step S144). In this case, if patient data is present, the presence of a medical appointment is confirmed. If there is a medical appointment, the visitor confirms the content of the appointment. If there is no medical appointment, the visitor enters the content of the medical questionnaire (Step S146). Through the above, the reception processing ends.

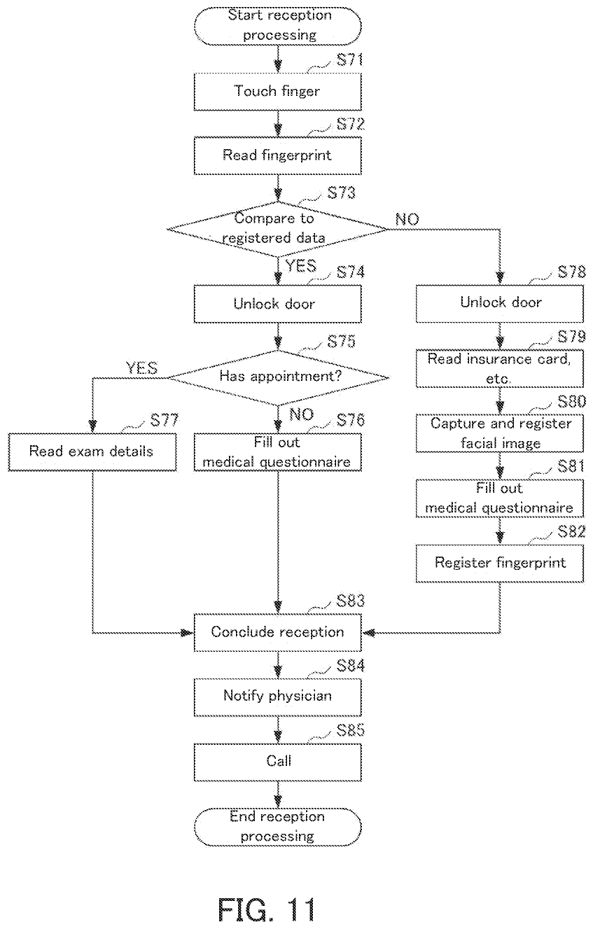

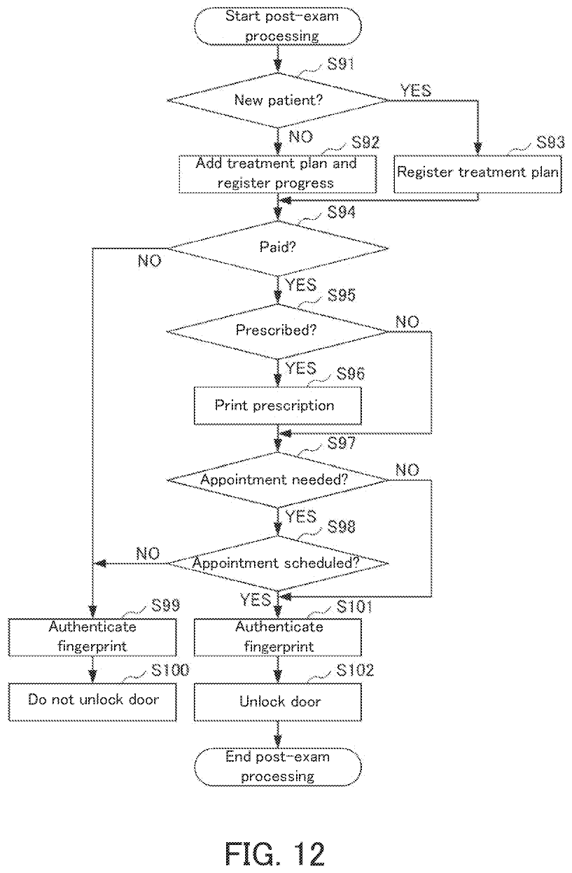

The patient authentication system (system in which a biometric terminal operates) is not limited to a facial authentication system, but may also use biometric authentication such as a fingerprint authentication system or an iris authentication system (not illustrated), as depicted for example in the flowcharts of FIGS. 11 and 12 as below.

FIG. 11 is a flowchart depicting the reception processing using a fingerprint authentication system.

As depicted in FIG. 11, the access management system can perform the reception processing using a fingerprint authentication system at the entrance of a medical institution or an examination room.

That is, when the reception processing using the fingerprint authentication system is started, the visitor touches their finger to the reception terminal 2 (Step S71). Then, the reception terminal 2 reads the fingerprint (Step S72) and compares the fingerprint to registered data (Step S73). If the fingerprint matches (that is, YES in Step S73), the door is unlocked (Step S74) and the presence of an appointment is confirmed (Step S75). When an appointment is present (that is, YES in Step S75), the examination content is read (Step S77), and the reception is completed (Step S83).

When the fingerprint does not match as a result of comparison to the registered data (Step S73) (that is, NO in Step S73), the door is unlocked (Step S78) and items such as an insurance card are read (Step S79). After that, an image of the visitor's face is captured and registered (Step S80), the medical questionnaire is filled out (Step S81), the fingerprint is registered (Step S82), and the reception is completed (Step S83).

Once the reception is complete, a physician is notified of the content of the examination (Step S84). When it is the patient's turn to be examined, a call is made (Step S85). Through the above, the reception processing ends.

FIG. 12 is a flowchart depicting the flow of processing for performing a post-examination procedure (hereinafter referred to as “post-examination processing”) performed in the medical administrative support equipment.

When the examination is for a new patient (that is, YES in Step S91), a treatment plan is registered (Step S93), and the processing advances to Step S94.

By contrast, when the examination is not for a new patient (that is, NO in Step S91), a treatment plan is added and a progress status is registered (Step S92). The processing then advances to Step S94.

In Step S94, a determination is made as to whether or not the medical fee has been paid. When the medical fee has been paid (that is, YES in Step S94), the processing advances to Step S95.

On the other hand, when the medical fee has not been paid (that is, NO in Step S94), the automatic door will not be unlocked (Step S100) even if fingerprint authentication has been performed in front of the automatic door (Step S99).

In Step S95, a determination is made as to whether or not medication has been prescribed. When medication has been prescribed (that is, YES in Step S95), a prescription is printed (Step S96) and the processing advances to Step S97.

By contrast, when no medication has been prescribed (that is, NO in Step S95), the processing advances to Step S97.

In Step S97, a determination is made as to whether or not an appointment for a subsequent examination is necessary. When an appointment for a subsequent examination is necessary (that is, YES in Step S97), the processing advances to Step S99.

On the other hand, when an appointment for a subsequent examination is not necessary (that is, NO in Step S97), the processing advances to Step S101.

In Step S98, a determination is made as to whether or not an appointment for a subsequent examination has been registered. When an appointment for a subsequent examination has already been registered (that is, YES in Step S98), the processing advances to Step S101.

On the other hand, when an appointment for a subsequent examination has not yet been registered (that is, NO in Step S98), the automatic door will not be unlocked (Step S100) even if fingerprint authentication is performed in front of the automatic door (Step S99).

In Step S101, fingerprint authentication is performed in front of the automatic door. If authentication is successful, the automatic door is unlocked (Step S102). Through the above, the post-examination processing ends.

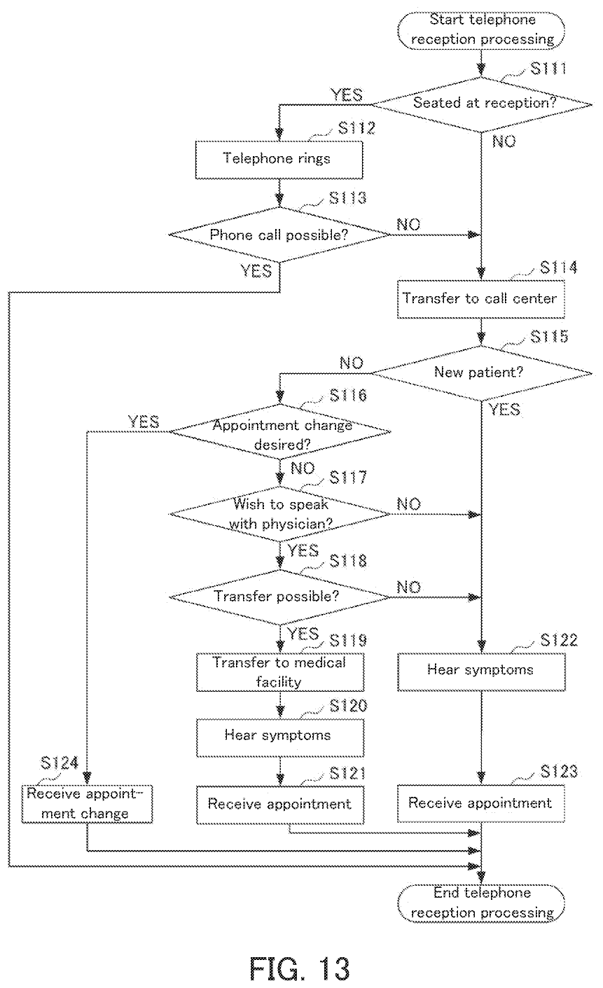

FIG. 13 is a flowchart depicting processing when reception processing is performed through a telephone (hereinafter referred to as “telephone reception processing”).

First, with a staff member present at the reception of the medical facility (that is, YES in Step S111), a reception telephone rings (Step S112). If the reception staff member is able to answer the telephone (that is, YES in Step S113), the reception staff member answers the telephone. Through the above, the telephone reception processing ends. On the other hand, when the reception staff member is unable to answer the telephone (during customer service, for example) (that is, NO in Step S113), the call is transferred to a call center (Step S114).

When no staff member is present at the reception of the medical facility (that is, NO in Step S111), the call is transferred to the call center (Step S114).

The call center confirms whether the caller is a first-time patient or not (Step S115). When the caller is a first-time patient (that is, YES in Step S115), the caller's symptoms are heard (Step S122) and an appointment is accepted (Step S123). Through the above, the telephone reception processing ends.

On the other hand, when the caller is not a first-time patient (that is, NO in Step S115), confirmation is made as to whether the subject of the call is to change an appointment (Step S116). When the subject of the call is to change an appointment (that is, YES in Step S116), an appointment change is accepted (Step S124). Through the above, the telephone reception processing ends.

When the subject of the call is not to change an appointment (that is, NO in Step S116), the caller is asked if they wish to speak with a physician (Step S117). When the caller does not wish to speak with a physician (that is, NO in Step S117), the call center representative hears the caller's symptoms (Step S122), and an appointment is accepted (Step S123). Through the above, the telephone reception processing ends.

When the caller wishes to speak with a physician (that is, YES in Step S117), a confirmation is made as to whether or not a medical facility, including a physician, is available (Step S118). When a medical facility is not available (that is, NO in Step S118), the call center representative hears the caller's symptoms (Step S122) and an appointment is accepted (Step S123). Through the above, the telephone reception processing ends.

If a medical facility including a physician can respond to the patient's request (that is, YES in Step S118), the call is transferred to the medical facility (Step S119), a reception staff member or a physician hears the patient's symptoms (Step S120), and an appointment is accepted (Step S121). Through the above, the telephone reception processing ends.

In this way, information from the call center is also spread to the medical care terminal 3. In addition, when urgent contact is needed, it is possible to switch to a telephone that can talk directly to the patient. When no reception staff member is available, a terminal (not illustrated) installed in the call center can detect a visitor using items such as a heat sensor and perform reception work on behalf of the reception staff member. This includes accepting changes of an appointment, and appointment information in the appointment system can also be changed via the Internet. In addition, messages can be sent to other terminals such the medical care terminal 3 and the administration terminal 5 when there is a complaint or an urgent message from a visitor or the like. In addition, it is possible to remotely turn on (ON) or off (OFF) the microphone function of a tablet of the medical care terminal 3.

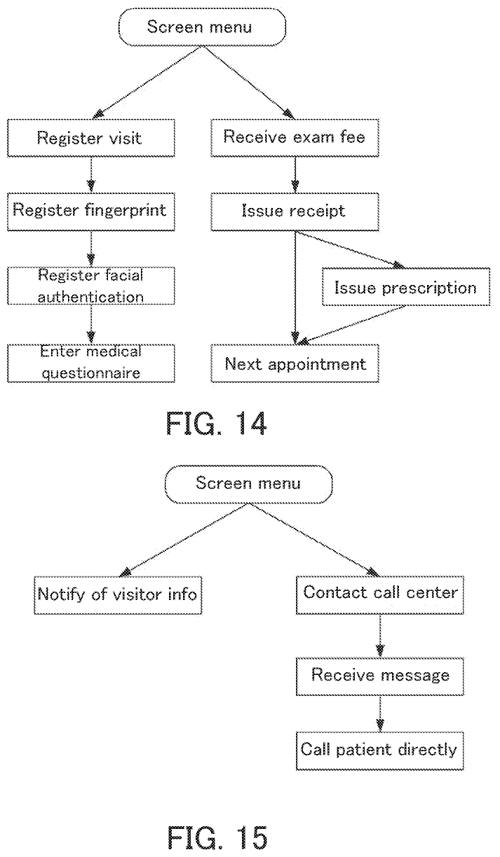

Next, the flow of processing performed at each terminal constituting the medical administrative support equipment G is described with reference to FIGS. 14 to 17.

As mentioned above, the reception terminal 2 and the procedure terminal 4 in FIG. 1 are terminals for a patient to perform the reception procedure and the post-examination procedure, respectively, but the reception terminal 2 and the procedure terminal 4 can also be a single terminal capable of performing both reception and post-examination procedures.

FIG. 14 is a flowchart depicting the flow of processing performed at a terminal capable of accepting both the reception procedure and the post-examination procedure (hereinafter referred to as a “multi-terminal”).

As illustrated in FIG. 14, the multi-terminal is triggered by an input operation by a patient on a screen menu, and processing of “register visit” or “receive examination fee” is performed.

In “register visit”, when the target of the visitor registration is a first-time patient, a fingerprint is registered, a captured facial image for facial authentication is registered, and input of a medical questionnaire is accepted.

In “receive medical fee”, when the payment of a medical fee is confirmed, a receipt is issued and an appointment for a subsequent examination is accepted. When medication is prescribed, a prescription is issued and an appointment for a subsequent examination is accepted.

FIG. 15 is a flowchart depicting the processing performed by the medical care terminal.

As depicted in FIG. 15, the medical care terminal 3 is triggered by an input operation by a physician on a screen menu, and processing of “notify of visitor information” or “contact call center” is performed.

In “notify of visitor information”, notification of information about a visitor is performed.

In “contact call center”, a message is received from the call center and a direct call from the patient is answered.

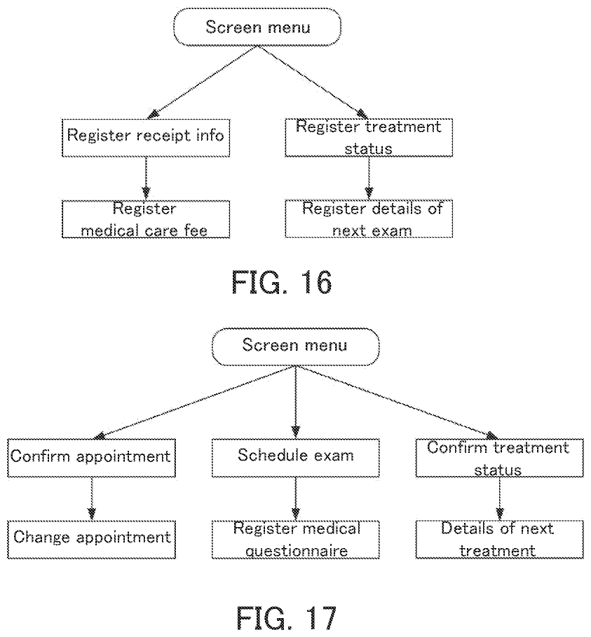

FIG. 16 is a flowchart depicting the processing performed by the administration terminal.

As depicted in FIG. 16, the administration terminal 5 is triggered by an input operation by an administrative staff member on a screen menu, and processing of “register receipt information” or “register treatment status” is performed.

In “register receipt information”, information on a receipt created based on an electronic chart is registered, as well a medical fee.

In “register treatment status”, information about the patient's treatment status is registered, and the content of the next examination is registered.

FIG. 17 is a flowchart depicting the processing performed by the patient terminal.

As depicted in FIG. 17, the patient terminal 8 is triggered by an input operation by the patient on a screen menu, and processing of “confirm appointment”, “schedule examination”, or “confirm treatment status” is performed.

In “confirm appointment”, the content of an appointment is displayed. When the patient wishes to change the appointment, an appointment change is registered.

In “schedule examination”, an appointment for a medical examination is accepted. In the case of a new patient, a medical questionnaire is registered.

In “confirm treatment status”, information indicating the treatment status is displayed, and information indicating subsequent treatment is displayed.

According to the medical administrative support equipment G that performs the above processing, the following specific services can be provided.

For example, it is assumed that a patient A, who has a history of visiting a dental clinic (medical institution) three months ago, is busy with work and interrupts treatment without notice, and returns to the clinic because pain has recurred in a tooth for which treatment was interrupted. Conventionally, the patient A would come directly to the dental clinic without even making a telephone appointment, hand over their patient registration card to reception, and complain about the pain or the like. Since the patient A would not have an appointment in advance, the reception staff member would search and browse through patient charts to obtain information about the patient A, who has come to the clinic with an emergency case. Only after entering the necessary patient data, the reception staff member could give a chart to a dentist or the dentist could view the chart. The dentist would then confirm the content of past examinations from the chart. In the meantime, the patient A would fill out necessary items such as a medical questionnaire in the waiting room. Alternatively, it would have taken time to confirm the patient data because the charts were searched and browsed after the patient A completed the medical questionnaire and the like.

By contrast, it is similarly assumed that the patient A comes directly to the dental clinic without even a telephone appointment. According to the present invention, when arriving at the clinic, the patient A would receive biometric authentication confirmation performed by a biometric authentication terminal installed next to the door of the dental clinic. Once the biometric authentication for the patient A is complete, the reception staff member in the clinic could access treatment data such as the patient's name, medical history, and interruption details. The dentist or the like could quickly confirm where the treatment was interrupted and where the pathology is present through the accessed treatment data. The patient A could then walk through the door of the dental clinic and wait in the waiting room. In other words, when the patient A waits in the waiting room, a medical administrative staff member such as the reception staff member and the dentist have already obtained the necessary information.

Conventionally, for example, it would have taken time to confirm patient A's chart and prepare for treatment. In addition, as for a dentist, the start of treatment would be delayed because the patient would need to be coached to be considerate of the dental clinic instead of visiting urgently after the affected area became painful. Therefore, the patient A feels badly for the dentist and the dental clinic.

By contrast, according to the present invention, since items such as the treatment plan for the patient A and sites with rates of emergency cases have been verified within the previous treatment period (including the first visit), the current emergency case arrival of the patient can be predicted to some extent. Therefore, the dentist can inform the patient A that the current emergency case is within the scope of prediction. As for the treatment itself, the dentist can start the treatment just like any other appointment. Even though the patient A is aware that they are coming to the dental clinic as an emergency case, they feel relieved and satisfied that the dental clinic has predicted the treatment and is practicing a quick and efficient flow. They also feel reassured that it is safe to come to the clinic next time for an emergency case.

Specifically, when the dentist knows of a part for which the treatment is not completed through checking an X-ray that has been taken, and also knows of the part that is likely to be the cause of an emergency case due to examination of the treatment. For example, when pain is expected in four out of eight teeth, it will be expected for the patient to returns as an emergency case. When the prediction is known by the system, both the dentist and the patient can feel relieved before treatment.

The entrance of a medical institution or an examination room functions as the exit of the medical institution or the examination room. Thus, in addition to the functions of access management, it is possible to settle medical expenses, issue prescriptions if necessary, and make subsequent appointments.

The progress of the patient's medical condition, including the treatment plan at the time of the first visit and the expected time of pain, is entered into the appointment system. The above information can also be viewed and confirmed by the patient, thus preventing interruptions in treatment.

The appointment system can also automatically search for the content of the next treatment and provide it to the patient. In addition, the appointment system can calculate the duration of treatment interruptions. In addition, the system can save the content of a day's appointments in Excel or other formats, so that they can be confirmed even in places without Internet access.

For example, conventionally, after treatment by the dental clinic, the patient A would have to go through reception to pay for treatment, receive a prescription, and make a subsequent appointment. However, since the reception processing was done by the reception staff member (human resources), the processing speed was limited. In addition, when the telephone rang, it would take time for the staff member to answer the telephone. In addition, there was a possibility that the patient A would disadvantage the dental clinic by being able to freely come and go from the dental clinic without paying the treatment fee or making a subsequent appointment.

By contrast, according to the present invention, the patient A can go to the procedure terminal 4 after treatment without going through the reception. At the procedure terminal 4, the patient can pay for the day's treatment, receive a prescription, and make a subsequent appointment. In the unlikely event that the patient A does not pay for the treatment or make an appointment, the warning device 7 will cause the door of the exit (entrance and exit) of the dental clinic to remain closed. At the same time or separately, an alarm sound may be emitted, or the warning content may be announced by voice.

The appointment system is not limited to a terminal installed in the medical institution, and a patient terminal 8 owned by the patient may also access the appointment system. From the patient terminal 8, it is possible to confirm and change an appointment, make an examination appointment, register medical details, confirm treatment status, and confirm the content of subsequent treatment.

From the patient terminal 8, it is possible to make a new appointment or change a previously set appointment. For example, it is possible to select the treatment time and physician and make an appointment.

Furthermore, when another patient cancels an appointment, other patients (users) can confirm appointment availability at the time of the cancellation, thus reducing the risk of time loss due to the appointment cancellation.

In addition, since the appointment system (reception system) is available 24 hours a day, patients (users) who have pain in the middle of the night can make an appointment immediately, which has the advantage of allowing them to calm down somewhat.

In addition, patient data is sent to the medical care terminal 3 through the appointment system, as patients are specified by authentication when they enter the clinic. The medical care terminal 3 can receive all of the information in real time and can switch between the information and display it as needed.

After a patient is authenticated by the patient authentication system, the medical care terminal 3 displays the respective information of first visit, interruption, and appointment. In other words, the medical care terminal 3 is notified of necessary visitor information. The term “interruption” refers to a state in which complete recovery from disease during the initial examination and ongoing treatment has not been confirmed. Therefore, in the case of a patient whose treatment has been interrupted, if a disease has been diagnosed by a physician, the disease of which treatment has been interrupted will be re-examined By contrast, if the patient whose treatment has been interrupted has a disease that is not yet diagnosed by the physician, the disease will be examined as a new disease. In some cases, a disease for which treatment has been interrupted and a new disease are examined simultaneously.

In recent years, it has become desirable to provide a system that makes it easier for patients who interrupt their treatment to return. Normally, 10% of patients cancel their treatment in a dental clinic every day. Sudden cancellations leave the dental clinic empty and unable to earn any remuneration from treatment. In addition, a patient who suddenly canceled treatment would be less likely to go for further treatment out of remorse (guilty feeling). Such sudden cancellation is one of the top reasons why patients who interrupt their treatment do not return.

This loss is even greater because not only the opportunity to earn treatment remuneration is lost due to sudden cancellations, but the opportunity for ongoing treatment is also lost.

Therefore, because the appointment system can be operated 24 hours a day, appointment times can be changed without hesitation not only from a personal computer but also from a mobile information terminal such as a smartphone (the patient terminal 8 in FIG. 1, for example), and cancellations without notice can be prevented.

By contrast, a patient feels pain after a while because treatment has been interrupted. Therefore, there is a possibility that the patient will return, but the possibility of having an emergency case is higher. At the first visit to the dental clinic, treatment cannot start smoothly unlike with an appointment (normal treatment).

In addition, a patient who abruptly canceled the above-mentioned appointment was likely to switch to another dental clinic for treatment. At the other dental clinic, it was necessary to create a new chart and treat the part that had been partially treated by the other dentist.

Therefore, it is necessary to avoid this kind of behavior.

As mentioned above, in order for a patient who suddenly cancels an appointment to return, it is important to dispel the feeling of remorse (guilt). Specifically, it is beneficial to explain a “system that facilitates the return of patients with interrupted treatment” in advance at the first visit because items such as the current treatment status and remaining treatment plans can be confirmed through the appointment system and appointments can be made 24 hours a day.

The appointment system manages appointments. The appointment system has a management function which handles problems such as forgetting to write, erase, or move an appointment, or having only one appointment book.

According to the medical administrative support device G, it is also possible to perform management for each of dental examination chairs (chairs), dentists, and staff members. Specifically, whether an appointment is available or not can be instantly checked based on three factors: the chairs, the attendance status of the dentists and the staff members, and the opening status of the clinic. Thus, double-booking of patients (users) can be prevented. In addition, since appointments can be managed in 10-minute increments for example, it is possible to realize a schedule with no waste.

In addition, if a patient (user) becomes busy for any reason, the appointment can be changed via the Internet on a mobile phone or a PC, eliminating the burden of rescheduling the appointment or contacting reception.

It is possible to provide an appointment confirmation function for patients (users) with appointments to confirm their appointments by sending out emails to the patients at predetermined timings before their respective examination times. By counting the number of times appointments are changed or canceled and the number of patients (users) who do not come as scheduled, it is possible to respond flexibly, set appointments carefully, and make appointments that take into account patients' circumstances.

By accumulating information related to appointments, such as the appointment status and visit history of patients (users), an appointment status list screen can be color-coded according to each staff member or physician in charge, and according to patient category. As a result, the system is very useful for smoother clinic management and better patient care.

In addition, according to the medical administrative support equipment G, the following problems and issues can be solved, for example.

In recent years, paperless and cashless systems have been promoted in various fields. However, the cost of going paperless and cashless is not borne by the customer (patients, for example), but by the party that provides the product or service to the customer (a medical facility, for example).

For a specific example, credit card payment at a medical facility is one way to go cashless, but this arrangement requires the medical facility to pay a predetermined percentage of the payment amount as an agency fee. This is not limited to credit cards, and the same arrangement is employed for smart payments with for example IC cards that can be used in transportation and smartphones.

As such, a problem arises that parties who provide products and services to customers have to bear all of the costs that may incur from going paperless and cashless.

Conventionally, payment of medical fees and other charges in a medical facility is made using a predetermined payment device (conventional product). This payment device (conventional product) calculates the fee amount to be paid by a patient based on the information obtained from a receipt.

However, this payment device (conventional product) is required to coordinate with a company that inspects receipts as an agent (hereinafter referred to as “receipt company”). Furthermore, many receipt companies are said to have exclusive rights to sell respective payment devices (conventional products).

As a result, payment devices (conventional products) are sold at high prices by receipt companies and are not widely used by the general public, causing a problem for manufacturers of payment devices (conventional products) in that they cannot increase their sales volume.

Conventionally, a medical facility totals daily revenue after business hours, but sometimes the amount of revenue is inconsistent. In such a case, the medical facility will have the person in charge of totaling work overtime to reconfirm the amount of revenue.

Thus, the medical facility is faced with the problem of having to pay overtime to the person in charge of totaling in a case in which the amount of revenue is inconsistent. As such, there is the desire to establish an arrangement which reduces the risk of such a situation occurring.

As to these problems, the medical administrative support equipment G in FIG. 1 can solve the above problems by means of a charge function and the like.

A “charge function” refers to a function that allows a patient to manage a predetermined amount of cash in their possession by the medical administrative support equipment G and use the cash to pay medical fees or the like at any time as long as it is within the predetermined amount (deposit money). With the charge function, patients can make cashless payments of medical service fees or the like as long as it is within an amount charged to the procedure terminal 4 in advance. In addition, the medical facility can reduce the costs associated with going cashless because cash management is facilitated and there is no need to bear agency fees such as with credit card payment, for example.

Specifically, when a physician completes an input operation for an electronic chart in a medical facility, information indicating items such as the treatment content and the medical fee are transferred from the medical care terminal 3 to the administration terminal 5. The person in charge of operating the administration terminal 5 issues an invoice or receipt (hereinafter referred to as an “invoice or the like”) as needed, and enters the settlement amount on the invoice or the like or scans the invoice or the like to read the settlement amount. Through the above, information indicating the settlement amount is transferred to a dedicated cloud service (hereinafter referred to as a “cloud”) provided by the medical server 1.

When the information indicating the settlement amount is transferred from the administration terminal 5, the medical server 1 (cloud) generates settlement data based on this information. In addition, an identifier such as a QR code (registered trademark) or barcode (hereinafter referred to as an “identification code”) is generated to uniquely identify the generated settlement data. The generated identification code is presented to the patient terminal 8 from the medical server 1 (cloud).

The patient terminal 8 can display the identification code presented from the medical server 1 (cloud) on a screen based on operation by the patient. When the patient displays the identification code on the screen of the patient terminal 8 and holds the identification code over the procedure terminal 4 to read it, the settlement amount is deducted from the charged amount. This allows the patient to make cashless payments for medical fees or the like.

The above is an example of cashless payment of medical fees or the like by displaying an identification code on the patient terminal 8, but it is also possible to make cashless payment of medical fees or the like using for example an IC card (an IC card-shaped patient registration card, for example) without using the patient terminal 8.

In this case, the medical server 1 (cloud) manages the settlement data transferred from the administration terminal 5 in association with identification information such as a patient ID. An IC card in which the identification information such as the patient ID is stored is distributed to the target patient as a patient registration card or the like. When the patient holds the IC card-shaped patient registration card over the procedure terminal 4 to read it in the same manner as the patient terminal 8 displaying the identification code as described above, the settlement amount is deducted from the charged amount.

In addition, an identifier such as a patient ID can be used in common with multiple medical facilities. As a result, it is no longer necessary to manage patient registration cards issued by each medical facility as in the past, which prevents a patient from forgetting to bring their patient registration card, for example.

The patient terminal 8 can also have a Global Positioning System (GPS) function to specify where the patient is located in the medical facility.

The following describes the flow of the settlement processing performed by the medical administrative support equipment G having the charge function.

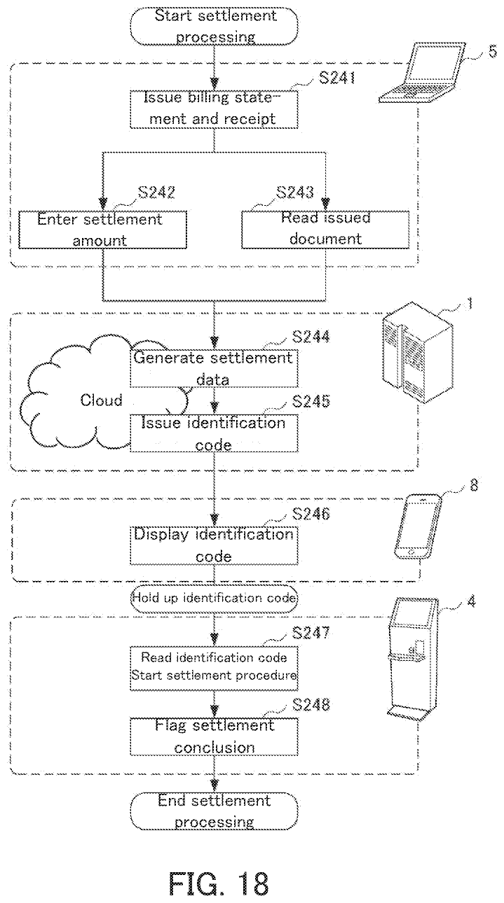

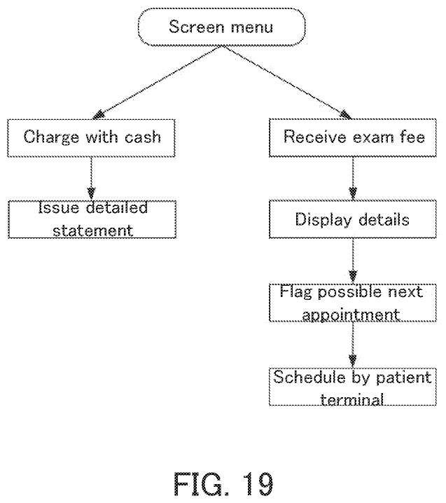

FIG. 18 is a flowchart depicting the settlement processing performed by the medical administrative support equipment of FIG. 1, which has the charge function.

In Step S241, the administration terminal 5 issues an invoice or the like as needed.

Upon performance of Step S241, either of Step S242 and Step S243 is performed.

In Step S242, the administration terminal 5 accepts an operation by which the settlement amount on the invoice or the like is entered and sends information indicating the accepted settlement amount to the medical server 1 (cloud).

In Step S243, the administration terminal 5 accepts an operation by which the invoice or the like is scanned, reads the settlement amount on the invoice or the like, and sends information indicating the read settlement amount to the medical server 1 (cloud).

In Step S244, the medical server 1 (cloud) generates settlement data based on the information indicating the settlement amount transferred from the administration terminal 5.

In Step S245, the medical server 1 (cloud) generates an identification code and presents the identification code to the patient terminal 8.

In Step S244, the medical server 1 (cloud) generates settlement data based on the information indicating the settlement amount transferred from the administration terminal 5.

In Step S245, the medical server 1 (cloud) generates and issues an identification code. The issued identification code is presented to the patient terminal 8.

In Step S246, the patient terminal 8 displays the identification code presented from the medical server 1 (cloud) on the screen based on an operation by the patient. The patient holds up the identification code displayed on the patient terminal 8 to the procedure terminal 4.

In Step S247, the procedure terminal 4 reads the identification code displayed on the screen of the patient terminal 8. This starts the settlement procedure.

In Step S248, the procedure terminal 4 flags the settlement as completed.

Through the above, the settlement process ends.

FIG. 19 is a flowchart depicting the processing performed by the procedure terminal that constitutes the medical administrative support equipment of FIG. 1 having the charge function.

As depicted in FIG. 19, the procedure terminal 4 is triggered by an input operation by a patient on a screen menu, and processing of “charge with cash” or “receive examination fee” is performed.

When the processing of charging cash takes place, a detailed statement is issued.

Once the examination fee has been paid from the charged cash amount, a detailed statement is issued. After that, a flag is set indicating that an appointment for a subsequent examination is available, and the appointment can be made at the patient terminal 8.

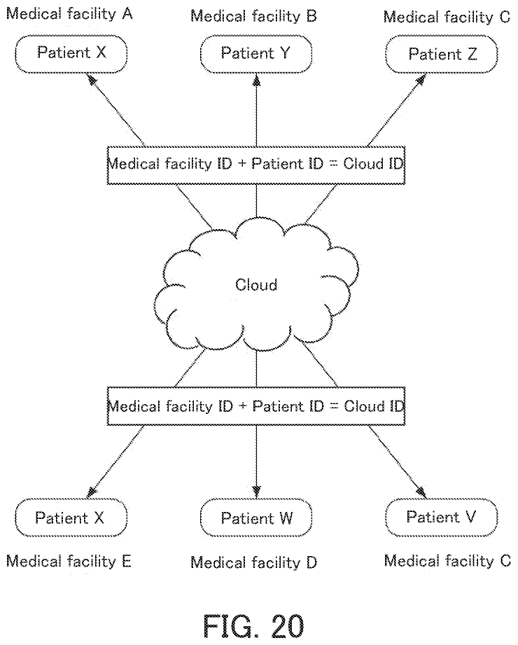

FIG. 20 is an image diagram illustrating the content of the cloud ID managed by the cloud provided by the medical server in FIG. 1.

In the cloud, management is performed using a cloud ID, which is a combination of a patient ID and a medical facility ID as identification information that can uniquely specify the medical facility.

For a specific example, as illustrated in FIG. 20, a cloud ID including a combination of a medical facility ID indicating a medical facility A and a patient ID indicating a patient X is managed by the cloud.

A cloud ID including a combination of a medical facility ID indicating a medical facility B and a patient ID indicating a patient Y is managed by the cloud.

A cloud ID including a combination of a medical facility ID indicating a medical facility C and a patient ID indicating a patient Z is managed by the cloud.

A cloud ID including a combination of a medical facility ID indicating a medical facility E and a patient ID indicating a patient X is managed by the cloud.

A cloud ID including a combination of a medical facility ID indicating a medical facility D and a patient ID indicating a patient W is managed by the cloud.

A cloud ID including a combination of a medical facility ID indicating a medical facility C and a patient ID indicating a patient V is managed by the cloud.

In this example, when the patient ID indicating the patient X is “0001”, the medical facility ID indicating the medical facility A is “0A”, and the medical facility ID indicating the medical facility E is “0E”, then a cloud ID “0A0001” and a cloud ID “0E0001” are both managed by the cloud. However, since these two cloud IDs are both cloud IDs for patient the X, they are associated with each other. As a result, payment of medical fees or the like for an individual patient is managed by the patient IDs (both are the same: “0001”), while the management of the medical facilities is managed by the medical facility IDs (“0A” and “0E”).

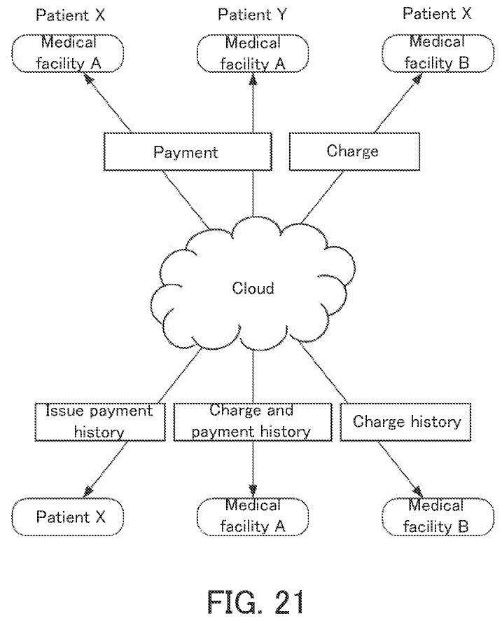

FIG. 21 is an image diagram illustrating a concrete example of a service that can be realized by the cloud.

As illustrated in FIG. 21, for example, after charging at the medical facility B, the patient X can pay the medical fee to medical facility A within the amount charged at medical facility B, even when they receive an examination at medical facility A.

In this case, the cloud presents a history in which the patient X has charged at medical facility B to both the medical facility B and the medical facility A. Also, the payment history of the patient X is presented to the patient X.

Thus, in the cloud, patients can use multiple medical facilities and their history is managed.

In this way, when a medical facility where charging was performed (the medical facility B in the above example) is different from a medical facility where the examination was made and the medical fee was incurred (the medical facility A in the above example), the charge and the medical fee offset each other between these two medical facilities. For a specific example, “processing for offsetting” is performed monthly for each of the medical facilities in batches, and each of the medical facilities is notified of the result of the processing.

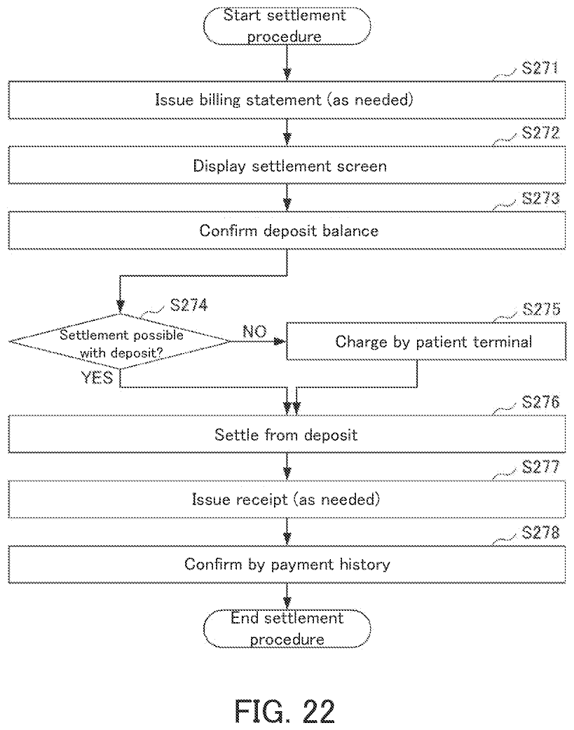

FIG. 22 is a flowchart depicting the flow of the settlement procedure by a patient after completing an examination.

As depicted in FIG. 22, in Step S271, the patient requests the issuance of a bill, if necessary, by operating the procedure terminal 4.

In Step S272, the patient operates the procedure terminal 4 to display an operation screen for performing the settlement procedure.

In Step S273, the patient operates the procedure terminal 4 to display and confirm the balance of deposit money (charged amount).

In Step S274, the patient determines whether or not settlement is possible with the balance of deposit money (charged amount). When the settlement is possible with the balance of deposit money (charged amount), a “YES” determination is made in Step S274 and the settlement procedure advances to Step S276. On the other hand, when the settlement is not possible with the balance of deposit money (charged amount) (insufficient balance or the like), a “NO” determination is made in Step S274 and the settlement procedure advances to Step S275.

In Step S275, the patient performs charging by operating the procedure terminal 4 and the patient terminal 8. At this time, the patient proves their identity in personal authentication by displaying an identification code on the patient terminal 8.

In Step S276, the patient performs the settlement procedure with the deposit money (charged amount) by operating the procedure terminal 4.

In Step S277, the patient requests the issuance of a receipt, if needed, by operating the procedure terminal 4.

In Step S278, the patient confirms the payment history by operating the procedure terminal 4.

Through the above, the settlement procedure by the patient ends.

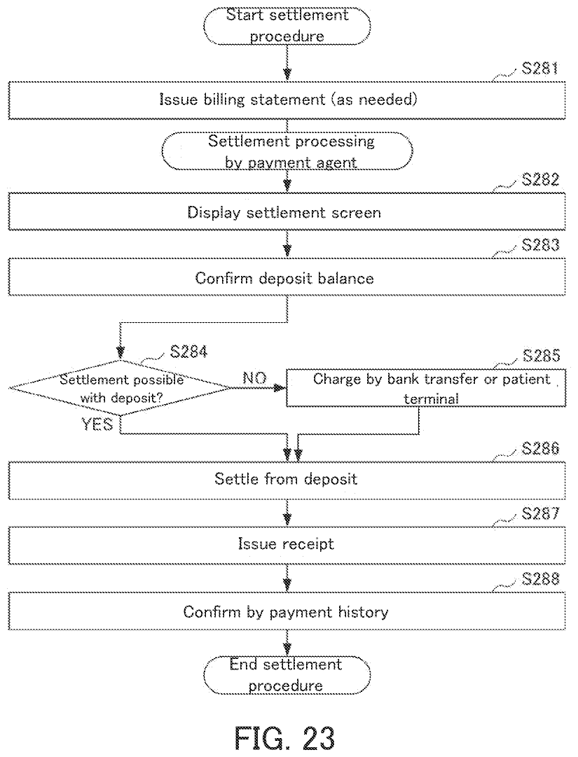

FIG. 23 is a flowchart depicting the flow of the settlement procedure when a patient who has completed an examination is a child or when the patient has a guardian.

As depicted in FIG. 23, in Step S281, the patient requests the issuance of a bill, if needed, by operating the procedure terminal 4.

Here, when the patient who has completed the examination is a child, or when the patient has a guardian, the settlement processing is performed by a person responsible for payment rather than by the patient themselves.

In Step S282, the person responsible for payment for the patient operates the procedure terminal 4 to display an operation screen for performing the settlement procedure.

In Step S283, the person responsible for payment for the patient operates the procedure terminal 4 to display and confirm the balance of deposit money (charged amount).

In Step S284, the person responsible for payment for the patient determines whether or not settlement is possible with the balance of deposit money (charged amount). When settlement is possible with the balance of deposit money (charged amount), a “YES” determination is made in Step S284 and the settlement procedure advances to Step S286. By contrast, when the settlement is not possible with the balance of deposit money (charged amount) (insufficient balance or the like), a “NO” determination is made in Step S284, and the settlement procedure advances to Step S285.

In Step S285, the person responsible for payment for the patient performs charging by making a bank transfer or by operating the procedure terminal 4 and the patient terminal 8. At this time, the person responsible for payment for the patient proves the identity of the individual (patient) in personal authentication by displaying an identification code on the patient terminal 8.

In Step S286, the person responsible for payment for the patient performs the settlement procedure with the deposit money (charged amount) by operating the procedure terminal 4.

In Step S287, the person responsible for payment for the patient requests the issuance of a receipt by operating the procedure terminal 4.

In Step S288, the person responsible for payment for the patient confirms the payment history by operating the procedure terminal 4.

Through the above, the settlement processing performed by the person responsible for payment for the patient ends.

The following describes the settlement procedure in a nursing home.

The types of medical care in nursing homes can be broadly divided into visiting care and outpatient care.

Conventionally, each of a visiting physician, a nursing home, and a relative of a resident would have to manage the payment of medical expenses and the medical history.

On this point, according to the above-mentioned embodiment, the visiting physician does not have to bring out patient information based on outpatient treatment on paper media because the information on the medical treatment (receipt) and the payment amount are uploaded to the cloud, which reduces the burden during an examination and prevents leakage of the patient's personal information. In addition, the visiting physician would need to send invoices and receipts to nursing homes or relatives through the physician's own clinic in order to receive payment for the medical treatment, but since these are managed in the cloud, the manpower to create invoices and receipts and the postage to send them can be reduced.

By contrast, nursing homes no longer have the burden of individually managing which residents have made which payments and received which medical treatments.

In particular, in the case of outpatient treatment, payment must be done at the hospital, so a patient has to do such things including estimation of the cost of the treatment and depositing cash in the estimated amount on hand from the patient's relative beforehand, which has caused not only a management burden but also a cash management burden. However, this can be avoided by using the above-mentioned charging function. For a specific example, by setting up a terminal in the nursing home to charge the cost of medical treatment, relatives can use the terminal to charge a patient terminal when they visit the facility, thereby eliminating the need to estimate the cost of medical treatment and have the relative deposit the money in the facility. In addition, the relatives will be relieved of the burden of having to visit the facility to pay for medical treatment.

An embodiment of the present invention is described above, but the present invention is not limited to the above-mentioned embodiment. The present invention includes any alteration, improvement, and the like within the scope of achieving the purpose of thereof. In addition, the effects described in the above embodiment are merely a list of the most favorable effects arising from the present invention. The effects of the present invention are not limited to what is described in the above-mentioned embodiment.

For example, in the above embodiments, the description is based on the assumption that the service is targeted at medical facilities and patients using the medical facilities, but it is not limited to the medical facilities and the patients using the medical facilities. The present invention can also be applied to any facilities and the like where goods or services are provided, as well as to services targeting users of such facilities.

The series of processing in the embodiment described above can be executed by hardware or software.

When the series of processing is to be executed by software, a program that constitutes the software is installed on a computer or the like through a network or storage medium.

The computer may be a computer that is embedded in dedicated hardware. Alternatively, the computer may be a computer capable of performing various functions through installation of various programs, such as a general-purpose personal computer.

The storage medium containing such a program includes not only removable media distributed separately from the main body of a device so to provide the program to the user, but also of storage media or the like with the program that are pre-embedded in the main body of a device provided to the user. Examples of removable media include magnetic disks (including a floppy disk), optical disks, and magneto-optical disks. Examples of the optical disks include compact disk read-only memory (CD-ROM) and a digital versatile disk (DVD). Examples of the magneto-optical disks include a minidisk (MD). Examples of the storage media that is pre-embedded in the main body of a device provided to the user include ROM in which the program is recorded and a hard disk included in storage.

In the present specification, steps describing the program recorded on a storage medium include not only processing performed chronologically in the order thereof, but also processing performed in parallel or individually without necessarily being processed chronologically.

In the present specification, the term “system” is assumed to mean a complete device including a plurality of devices, a plurality of means, and the like.

The system configuration illustrated in FIG. 1 is merely an example to achieve the object of the present invention and is not a particular limitation. In other words, it is sufficient if functions capable of performing the roles of respective devices are provided in the information processing system, and furthermore, each device may be directly connected without going through a network.

Each hardware configuration illustrated in FIG. 2 is merely an example to achieve the object of the present invention and is not a particular limitation. For example, one piece of hardware may have the functions of other hardware, and multiple pieces of hardware with the same function may be included.

The functional configuration in FIG. 3 is only an example and is not a particular limitation. In other words, it is sufficient that the server 1 include functions capable of executing the series of processing described above as a whole, and the functional blocks which are used to realize these functions is not particularly limited to the example in FIG. 3.

A single functional block may be constituted by hardware alone, software alone, or a combination of the two.

In summary, the information processing device to which the present invention is applied should have the following configuration, and may be implemented in various embodiments.

That is, the information processing device (the medical server 1 in FIG. 1, for example) to which the present invention is applied includes:

a first management means (the queue management section 101 in FIG. 3, for example) which generates first information (queue data, for example) indicating an order in which customers receive provision of an object of goods or services at a facility where the object is provided and manages the first information for each customer;

an output means (the issuance controller 102 in FIG. 3, for example) which performs control to output a medium (a queue card, for example) indicating the first information to be presented to the customers;

a second management means (the location management section 103 in FIG. 3, for example) which manages second information (patient location information, for example) indicating a location in the facility of a customer among the customers possessing the medium in association with the first information; and

a third management means (the examination management section 104 in FIG. 3, for example) which manages third information (an electronic chart, for example) indicating content of the goods or services in association with the first information;

a fourth management means (the procedure management section 105 in FIG. 3, for example) which manages fourth information (procedure progress information, for example) indicating a progress status of a procedure (a “post-examination procedure”, for example) to be performed after the provision of the object in association with the first information, the procedure including settlement of an amount of a fee for the object determined based on information including at least the third information; and

a monitoring means (the monitoring section 106 in FIG. 3, for example) which performs monitoring as to whether or not a status of movement of the customer matches a procedure status based on the first information, the second information, the third information, and the fourth information.

In the above configuration, the first management means generates the first information indicating an order in which customers receive the provision of the object of goods or services at the facility where the object is provided and manages the first information for each customer, and the output means performs control to output a medium indicating the first information to be presented to the customers. The second management means manages the second information indicating a location in the facility of a customer among the customers possessing the medium in association with the first information, and the third management means manages the third information indicating content of the goods or services in association with the first information. In addition, the fourth management means manages the fourth information indicating a progress status of the procedure to be performed after the provision of the object of the goods or services in association with the first information, the procedure including settlement of an amount of a fee for the object of the goods or services determined based on information including at least the third information. In addition, the monitoring means performs monitoring as to whether or not a status of movement of the customer matches the procedure status based on the first information, the second information, the third information, and the fourth information.

As a result, it is possible to reduce the number of staff members and save labor in the procedures of the facility that provides goods or services without requiring a customer to operate a mobile information terminal.

In addition, the first management means generates, as the first information, information indicating an order in which patients receive medical treatment by a physician at a medical facility as the facility, and manages the first information for each patient.

The output means performs control to output a medium indicating the first information to be presented to the patients.

The second management means manages, as the second information, information indicating a location in the medical facility of a patient among the patients possessing the medium in association with the first information.

The third management means manages, as the third information, information indicating content of the medical treatment of the patient by the physician in association with the first information.

The fourth management means manages, as the fourth information, information indicating a progress status of a procedure to be performed after the medical treatment in association with the first information, the procedure including settlement of a fee for the medical treatment for which an amount is determined based on information including at least the third information.

The monitoring means can perform monitoring as to whether or not a status of movement of the patient matches the procedure status based on the first information, the second information, the third information, and the fourth information.

In the above configuration, it is possible to reduce the number of staff members and save labor in a reception procedure of a medical institution without requiring the patient to operate a mobile information terminal.

The information processing device may further include a warning control means (the warning controller 107 in FIG. 3, for example) which performs control to perform a predetermined warning to a corresponding patient among the patients when the status of the movement of the corresponding patient does not match the procedure status as a result of the monitoring by the monitoring means.

In the above configuration, misunderstanding and fraud by the patient can be prevented.

In addition, the fourth management means can manage, as the fourth information, information indicating presence or absence of a prescription in association with the first information.

In the above configuration, it is possible to reduce the number of staff members and save labor in various procedures of a medical institution, including receipt of prescriptions, without requiring the patient to operate a mobile information terminal.

In addition, the fourth management means can manage, as the fourth information, information indicating presence or absence of an appointment for subsequent medical treatment in association with the first information.

In the above configuration, it is possible to reduce the number of staff members and save labor in various procedures of a medical institution, including acceptance of appointments, without requiring the patient to operate a mobile information terminal.

In addition, the fourth management means can further manage fifth information (information indicating a charged amount of deposit money, for example) indicating an amount of deposit money deposited by each of the patients for the settlement of the fee for the medical treatment in association with sixth information (a patient ID, for example) including at least predetermined identification information capable of uniquely specifying a corresponding patient.

In the above configuration, the patient can complete payment of fees for medical treatment on a cashless basis, as long as it is within an amount of deposit money deposited in advance. In addition, the medical facility can reduce costs that may arise from going cashless, since cash management is facilitated and there is no need to bear agency fees such as with credit card payments, for example.

In addition, the fourth management means further manages seventh information (settlement data, for example) indicating the amount of the fee for the medical treatment determined based on information (a receipt, for example) including at least the third information (an electronic chart, for example) in association with the first information (queue data, for example).

The information processing device may further include a generating means (the code generator 108 in FIG. 3, for example) which generates an identifier (a QR code (registered trademark), for example) capable of uniquely specifying the seventh information managed by the fourth management means.

Through the above, the patient can perform cashless payment of fees for medical services or the like. In addition, the medical facility can easily cope with cashless and paperless systems.

### REFERENCE SIGNS LIST

S Medical administrative support equipment
2 Reception terminal
3 Examination terminal
4 Procedure terminal
5 Administration terminal
6 Card detector
7 Warning device
8 Patient terminal
1 Medical server
11 CPU
12 ROM
13 RAM
14 Bus
15 Input/output interface
16 Output section
17 Input section
18 Storage
19 Communication section
20 Drive
30 Removable media
101 Queue management section
102 Issuance controller
103 Location management section
104 Examination management section
105 Procedure management section
106 Monitoring section
107 Warning controller
108 Code generator
181 Patient DB
182 Card DB
S Step
N Communication network

1. An information processing device comprising:
a first management means configured to generate first information indicating an order in which customers receive provision of an object of goods or services at a facility where the object is provided and manage the first information for each customer;
an output means configured to perform control to output a medium indicating the first information to be presented to the customers;
a second management means configured to manage second information indicating a location in the facility of a customer among the customers possessing the medium in association with the first information;
a third management means configured to manage third information indicating content of the goods or services in association with the first information;
a fourth management means configured to manage fourth information indicating a progress status of a procedure to be performed after the provision of the object in association with the first information, the procedure including settlement of an amount of a fee for the object determined based on information including at least the third information; and
a monitoring means configured to perform monitoring as to whether or not a status of movement of the customer matches a procedure status based on the first information, the second information, the third information, and the fourth information.

2. The information processing device according to claim 1, wherein
the first management means generates, as the first information, information indicating an order in which patients receive medical treatment from a physician at a medical facility as the facility, and manages the first information for each patient,
the output means performs control to output a medium indicating the first information to be presented to the patients,
the second management means manages, as the second information, information indicating a location in the medical facility of a patient among the patients possessing the medium in association with the first information,
the third management means manages, as the third information, information indicating content of the medical treatment of the patient by the physician in association with the first information,
the fourth management means manages, as the fourth information, information indicating a progress status of a procedure to be performed after the medical treatment in association with the first information, the procedure including settlement of a fee for the medical treatment for which an amount is determined based on the information including at least the third information, and
the monitoring means performs monitoring as to whether or not a status of movement of the patient matches the procedure status based on the first information, the second information, the third information, and the fourth information.

3. The information processing device according to claim 2, further comprising
a warning control means configured to perform control to perform a predetermined warning to a corresponding patient among the patients when the status of the movement of the corresponding patient does not match the procedure status as a result of the monitoring by the monitoring means.

4. The information processing device according to claim 2, wherein
the fourth management means manages, as the fourth information, information indicating presence or absence of a prescription in association with the first information.

5. The information processing device according to claim 2, wherein
the fourth management means manages, as the fourth information, information indicating presence or absence of an appointment for subsequent medical treatment in association with the first information.

6. The information processing device according to claim 2, wherein
the fourth management means further manages fifth information indicating an amount of deposit money deposited by each of the patients for the settlement of the fee for the medical treatment in association with sixth information including at least predetermined identification information capable of uniquely specifying a corresponding patient.

7. A non-transitory computer readable storage medium in which a program is recorded, the program causing a computer controlling an information processing device to perform control processing, the control processing comprising:
a first management step of generating first information indicating an order in which customers receive provision of an object of goods or services at a facility where the object is provided and managing the first information for each customer;
an outputting step of performing control to output a medium indicating the first information to be presented to the customer.
a second management step of managing second information indicating a location in the facility of a customer among the customers possessing the medium in association with the first information;
a third management step of managing third information indicating content of the goods or services in association with the first information;
a fourth management step of managing fourth information indicating a progress status of a procedure to be performed after the provision of the object in association with the first information, the procedure including settlement of an amount of a fee for the object determined based on the information including at least the third information; and
a monitoring step of performing monitoring as to whether or not a status of movement of the customer matches a procedure status based on the first information, the second information, the third information, and the fourth information.
